(19) United States
(12) Patent Application Publication
(10) Pub. No.: US 2009/0048233 A1
(43) Pub. Date: Feb. 19, 2009

(54) Enhancing the Tolerability of a Seratonin Antagonist and a NSRI, a SNRI or a RIMA by Using Them in Combination

(75) Inventors: Srinivas RAO, Encinitas, CA (US); Jay Kranzler, La Jolla, CA (US); Jeffery J. Anderson, San Diego, CA (US)

Correspondence Address: WILSON SONSINI GOODRICH & ROSATI, 650 PAGE MILL ROAD, PALO ALTO, CA, 94304-1050, US

(73) Assignee: CYPRESS BIOSCIENCES, INC., San Diego, CA (US)

(21) Appl. No.: 12/192,591

(22) Filed: Aug. 15, 2008

Related U.S. Application Data
(60) Provisional application No. 60/956,317, filed on Aug. 16, 2007.
(60) Provisional application No. 61/051,416, filed on May 8, 2008.
(60) Provisional application No. 61/075,246, filed on Jun. 24, 2008.

(51) Int. Cl. A61K 31/55; A61K 31/435; A61K 31/164; A61K 31/4525; A61K 31/5375; A61K 31/381; A61K 31/13; A61P 25/00

(52) U.S. Cl. 514/214.02; 514/284; 514/624; 514/320; 514/237.8; 514/438; 514/646

(57) ABSTRACT

Provided are combinations of pharmaceutical agents capable of eliciting a therapeutic effect. A first therapeutic agent in the combination has 5HT₂/5HT₃ antagonist and alpha-2 antagonist activity. A second therapeutic agent possesses both serotonin reuptake inhibitory activity and norepinephrine reuptake inhibitory activity or is a reversible inhibitor of monoamine oxidase A (RIMA). In some embodiments, a combination of a first therapeutic agent and a second therapeutic agent results in reduction in one or deleterious side effects associated with the first therapeutic agent, the second therapeutic agent or both. Also provided are methods of treatment employing a first therapeutic agent and a second therapeutic agent. Also provided are kits containing a first therapeutic agent, a second therapeutic agent and instructions for administering the first therapeutic agent and the second therapeutic agent.

This application claims the benefit of priority of U.S. Provisional Application No. 60/956,317, filed Aug. 16, 2007, U.S. Provisional Application No. 61/051,416, filed May 8, 2008, and U.S. Provisional Application No. 61/075,246, filed Jun. 24, 2008, each of which applications is expressly incorporated herein by reference in its entirety.

### BACKGROUND

1. Technical Field

The disclosure relates to certain combinations of drugs which confer at least one beneficial therapeutic effect on a patient, while simultaneously providing reduced incidence and/or severity of at least one averse effect associated with one or more of the drugs.

2. Technical Background

Mirtazapine has been utilized effectively in the treatment of depression. It is also effective in the treatment of schizophrenia, anxiety disorders, affective disorders, sleep apnea, insomnia, migraine headache, chronic tension-type headache, hot flashes, and fibromyalgia. Mirtazapine owes its diverse utility in treating this range of disorders to its diverse pharmacology. Mirtazapine acts as an antagonist at presynaptic alpha-2 (α₂) adrenergic receptors on both norepinephrine and serotonin (5-HT) presynaptic nerve terminals. In addition, it acts as a potent antagonist at 5HT_{2A} serotonin receptors, 5HT_{2C} serotonin receptors, 5HT₃ serotonin receptors, and histamine H1 receptors. Mirtazapine is a very weak inhibitor of norepinephrine reuptake and a weak antagonist at both muscarinic cholinergic and alpha-1 (α₁) adrenergic receptors, and has no effect on the reuptake of dopamine or 5-HT. The net outcome of these effects is increased noradrenergic and serotonergic activity, especially at 5HT_{1A} serotonin receptors. However, mirtazapine can produce side effects that lead to reduced efficacy, reduced patient compliance or both. The side effects may include marked gains in body weight and excessive daytime sleepiness or drowsiness. The weight gain is likely due to the 5HT_{2C} and H1 receptor antagonistic effects of mirtazapine, while the excessive daytime drowsiness is likely a result of H1 receptor antagonism.

Setiptiline ((1,2,3,4-tetrahydro-2-methyl-9H-dibenzo[3,4:6,7]cyclohepta[1,2-C]pyridine maleate)) is a drug having antagonist activity toward the central 5HT₂, 5HT₃ and α₂ receptors and possesses indications and pharmacology that are very similar to those of mirtazapine. Considering that the mirtazapine and setiptiline have similar microbiological activity, it is likely that setiptiline will have a side effect profile similar to that of mirtazapine.

The rates of obesity and overweight have increased drastically over the last decade; and there is a high prevalence of obesity in patients with mental illness. Hence, highly effective drugs like mirtazapine, and presumably setiptiline, which produce increases in appetite and body weight, may nonetheless present too great a risk for use in this patient population.

The excessive daytime drowsiness and mental impairment produced by mirtazapine can negatively impact driving and job performance. To reduce the propensity for drowsiness, mirtazapine is often administered at night. However, because of the long elimination T½ (20-40 hr) of this drug, drowsiness often occurs even the day following administration. A reduction in the incidence of these side effects (sedation and weight gain) would greatly enhance the effectiveness of mirtazapine pharmacotherapy.

Other antidepressant drugs, such as serotonin norepinephrine reuptake inhibitors (SNRIs), norepinephrine serotonin reuptake inhibitors (NSRIs), and reversible inhibitors of monoamine oxidase A (RIMAs); however these drugs also have negative side effects, such as nausea and emesis.

There is thus a need for compositions and methods of treating depression, schizophrenia, anxiety disorders, affective disorders, sleep-related breathing disorders, snoring, insomnia, migraine headache, chronic tension-type headache, hot flashes, chronic lower back pain, neuropathic pain (e.g. from diabetic peripheral neuropathy) and functional somatic syndromes.

### SUMMARY

The foregoing and further needs are met by embodiments described herein. The disclosure herein provides combinations of therapeutic agents having improved side effect profiles as compared to the therapeutic agents when dosed separately. In particular, the disclosure provides combinations comprising first and second therapeutic agents, wherein the first therapeutic agent is an antagonist of 5HT₂/5HT₃ and α₂ receptors and the second therapeutic agent is a catecholamine reuptake inhibitor or a reversible inhibitor of monoamine oxidase A. In some embodiments described herein, related advantages of the combination therapy described herein include a reduction in one or more of the side effects normally associated with administration of the catecholamine reuptake inhibitor or a reversible inhibitor of monoamine oxidase A.

Some embodiments disclosed herein meet one or more of the foregoing needs and/or provide additional or related advantages as well, by providing a method of reducing the incidence or severity of one or more side effects associated with administration of at least a first therapeutic agent, a second therapeutic agent or both in the treatment of a disorder in a patient, wherein the first therapeutic agent has 5HT₂/5HT₃ antagonist and alpha-2 antagonist activity, and the second therapeutic agent possesses both serotonin reuptake inhibitory activity and norepinephrine reuptake inhibitory activity or is a reversible inhibitor of monoamine oxidase A (RIMA), comprising administering to the patient an effective amount of the first therapeutic agent and the second therapeutic agent.

Some embodiments disclosed herein meet one or more of the foregoing needs and/or provide additional or related advantages as well, by providing a formulation comprising an effective amount of a combination of a first therapeutic agent and a second therapeutic agent, wherein the first therapeutic agent is a 5HT₂/5HT₃ antagonist/alpha-2 antagonist and the second therapeutic agent possesses both serotonin reuptake inhibitory activity and norepinephrine reuptake inhibitory activity or is a reversible inhibitor of monoamine oxidase A (RIMA).

Some embodiments disclosed herein meet one or more of the foregoing needs and/or provide additional or related advantages as well, by providing a method of treating a disorder treatable by administration of a first therapeutic agent having 5HT2/5HT3 antagonist and alpha-2 antagonist activity, a second therapeutic agent, or both, wherein the second therapeutic agent possesses both serotonin reuptake inhibitory activity and norepinephrine reuptake inhibitory activity, the method comprising administering the first therapeutic agent to the patient, and within about 18 hours of administering the first therapeutic agent, administering the second therapeutic agent, wherein combined administration of the first therapeutic agent and the second therapeutic agent is effective to treat at least one disorder, wherein a reduction in at least one side effect associated with the first therapeutic agent, the second therapeutic agent, or both is obtained, and wherein at least one such side effect is selected from the group consisting of daytime sedation, nausea, emesis, weight gain and cognitive impairment.

Some embodiments disclosed herein meet one or more of the foregoing needs and/or provide additional or related advantages as well, by providing a kit comprising a first therapeutic agent, a second therapeutic agent and instructions for administering the first therapeutic agent before bed and the second therapeutic agent after waking, wherein the first therapeutic agent comprises a 5HT₂/5HT₃ antagonist/alpha-2 antagonist and the second therapeutic agent possesses both serotonin reuptake inhibitory activity and norepinephrine reuptake inhibitory activity or is a reversible inhibitor of monoamine oxidase A (RIMA).

Some embodiments disclosed herein meet one or more of the foregoing needs and/or provide additional or related advantages as well, by providing a unit dosage form containing a synergistic combination of a first therapeutic agent and a second therapeutic agent, wherein the first therapeutic agent comprises a 5HT₂/5HT₃ antagonist/alpha-2 antagonist and the second therapeutic agent possesses both serotonin reuptake inhibitory activity and norepinephrine reuptake inhibitory activity norepinephrine or is a reversible inhibitor of monoamine oxidase A (RIMA).

Some embodiments disclosed herein meet one or more of the foregoing needs and/or provide additional or related advantages as well, by providing a combination of mirtazapine and milnacipran, either co-administered in a single unit dose (e.g. before bed, with mirtazapine as an immediate release form and milnacipran as a delayed release form; or after waking, with milnacipran as an immediate release form and mirtazapine as a delayed-release form), or as separately administered forms (e.g. mirtazapine before bed and milnacipran after waking), as well as methods of treating one or more disease states employing the combination of mirtazapine and milnacipran (e.g. as one of the aforementioned unit doses or as separate unit doses) and/or kits containing a combination of mirtazapine and milnacipran (e.g. as separate unit doses) for treating one or more disease states. In some embodiments, some portion (or in some cases all) of the dose of milnacipran may be replaced with bicifadine. Some embodiments described herein meet a long-felt need of reducing at least one negative side effect associated with solo administration of mirtazapine. In some embodiments, related advantages include reduction in at least one negative side effect associated with administration of bicifadine.

Some embodiments disclosed herein meet one or more of the foregoing needs and/or provide additional or related advantages as well, by providing a combination of mirtazapine and duloxetine, either co-administered in a single unit dose (e.g. before bed, with mirtazapine as an immediate release form and duloxetine as a delayed release form; or after waking, with duloxetine as an immediate release form and mirtazapine as a delayed-release form), or as separately administered forms (e.g. mirtazapine before bed and duloxetine after waking), as well as methods of treating one or more disease states employing the combination of mirtazapine and duloxetine (e.g. as one of the aforementioned unit doses or as separate unit doses) and/or kits containing a combination of mirtazapine and duloxetine (e.g. as separate unit doses) for treating one or more disease states. It in some embodiments, some portion (or in some cases all) of the dose of duloxetine may be replaced with venlafaxine, desvenlafaxine or a combination thereof. Some embodiments described herein meet a long-felt need of reducing at least one negative side effect associated with solo administration of mirtazapine. In some embodiments, related advantages include reduction in at least one negative side effect associated with administration of duloxetine.

Some embodiments disclosed herein meet one or more of the foregoing needs and/or provide additional or related advantages as well, by providing a combination of mirtazapine and brofaromine or moclobemide, either co-administered in a single unit dose (e.g. before bed, with mirtazapine as an immediate release form and brofaromine or moclobemide as a delayed release form; or after waking, with brofaromine or moclobemide as an immediate release form and mirtazapine as a delayed-release form), or as separately administered forms (e.g. mirtazapine before bed and brofaromine or moclobemide after waking), as well as methods of treating one or more disease states employing the combination of mirtazapine and brofaromine or moclobemide (e.g. as one of the aforementioned unit doses or as separate unit doses) and/or kits containing a combination of mirtazapine and brofaromine or moclobemide (e.g. as separate unit doses) for treating one or more disease states. Some embodiments described herein meet a long-felt need of reducing at least one negative side effect associated with solo administration of mirtazapine. In some embodiments, related advantages include reduction in at least one negative side effect associated with administration of brofaromine or moclobemide.

Some embodiments disclosed herein meet one or more of the foregoing needs and/or provide additional or related advantages as well, by providing a combination of setiptiline and milnacipran, either co-administered in a single unit dose (e.g. before bed, with setiptiline as an immediate release form and milnacipran as a delayed release form; or after waking, with milnacipran as an immediate release form and setiptiline as a delayed-release form), or as separately administered forms (e.g. setiptiline before bed and milnacipran after waking), as well as methods of treating one or more disease states employing the combination of setiptiline and milnacipran (e.g. as one of the aforementioned unit doses or as separate unit doses) and/or kits containing a combination of setiptiline and milnacipran (e.g. as separate unit doses) for treating one or more disease states. In some embodiments, some portion (or in some cases all) of the dose of milnacipran may be replaced with bicifadine. Some embodiments described herein meet a long-felt need of reducing at least one negative side effect associated with solo administration of setiptiline. In some embodiments, related advantages include reduction in at least one negative side effect associated with administration of bicifadine.

Some embodiments disclosed herein meet one or more of the foregoing needs and/or provide additional or related advantages as well, by providing a combination of setiptiline and duloxetine, either co-administered in a single unit dose (e.g. before bed, with setiptiline as an immediate release form and duloxetine as a delayed release form; or after waking, with duloxetine as an immediate release form and setiptiline as a delayed-release form), or as separately administered forms (e.g. setiptiline before bed and duloxetine after waking), as well as methods of treating one or more disease states employing the combination of setiptiline and duloxetine (e.g. as one of the aforementioned unit doses or as separate unit doses) and/or kits containing a combination of setiptiline and duloxetine (e.g. as separate unit doses) for treating one or more disease states. It in some embodiments, some portion (or in some cases all) of the dose of duloxetine may be replaced with venlafaxine, desvenlafaxine or a combination thereof. Some embodiments described herein meet a long-felt need of reducing at least one negative side effect associated with solo administration of setiptiline. In some embodiments, related advantages include reduction in at least one negative side effect associated with administration of venlafaxine.

Some embodiments disclosed herein meet one or more of the foregoing needs and/or provide additional or related advantages as well, by providing a combination of setiptiline and brofaromine or moclobemide, either co-administered in a single unit dose (e.g. before bed, with setiptiline as an immediate release form and brofaromine or moclobemide as a delayed release form; or after waking, with brofaromine or moclobemide as an immediate release form and setiptiline as a delayed-release form), or as separately administered forms (e.g. setiptiline before bed and brofaromine or moclobemide after waking), as well as methods of treating one or more disease states employing the combination of setiptiline and brofaromine or moclobemide (e.g. as one of the aforementioned unit doses or as separate unit doses) and/or kits containing a combination of setiptiline and brofaromine or moclobemide (e.g. as separate unit doses) for treating one or more disease states. Some embodiments described herein meet a long-felt need of reducing at least one negative side effect associated with solo administration of setiptiline. In some embodiments, related advantages include reduction in at least one negative side effect associated with administration of brofaromine or moclobemide.

### INCORPORATION BY REFERENCE

All publications and patent applications mentioned in this specification are herein incorporated by reference to the same extent as if each individual publication or patent application was specifically and individually indicated to be incorporated by reference.

### DETAILED DESCRIPTION

Disclosed herein are combinations of therapeutic agents having improved side effect profiles as compared to the therapeutic agents when dosed separately. In particular, the disclosure provides combinations comprising first and second therapeutic agents, wherein the first therapeutic agent is an antagonist of 5HT₂/5HT₃ and α₂ receptors and the second therapeutic agent is a catecholamine reuptake inhibitor or a reversible inhibitor of monoamine oxidase A. Catecholamine reuptake inhibitors taught herein include the serotonin norepinephrine reuptake inhibitors (SNRIs) and norepinephrine serotonin reuptake inhibitors (NSRIs). A SNRI is a therapeutic agent that inhibits reuptake of serotonin and, to a lesser extent, norepinephrine, thereby increasing the intrasynaptic concentration of both serotonin and norepinephrine. Some SNRIs may have additional activity, such as dopamine reuptake inhibitory activity, that may further affect their pharmacodynamic effects. A NSRI is a therapeutic agent that inhibits reuptake of norepinephrine and, to an equal or lesser extent, serotonin. Some NSRIs may also have additional activity, such as dopamine reuptake inhibitory activity. A RIMA is a monoamine oxidase inhibitor that is selective for the A isoenzyme and binds the enzyme reversibly. A RIMA may also have additional, less pronounced, activity that affects its pharmacodynamic profile. Also described herein are methods of using, as well as kits containing, the combinations described herein. The combinations, methods and kits provide improved treatment of one or more psychological or psychosomatic disorders as described in more detail herein. The improvement of treatment may include an improvement in one or more side effects associated with the first or second therapeutic agent. The improvement of treatment may also include an improvement in pharmacodynamic profile as compared to either therapeutic agent taken separately, and may include a synergistic effect.

Some embodiments disclosed herein provide a method of reducing the incidence or severity of one or more side effects associated with administration of at least a first therapeutic agent, a second therapeutic agent or both in the treatment of a disorder in a patient, wherein the first therapeutic agent has 5HT₂/5HT₃ antagonist and alpha-2 antagonist activity, and the second therapeutic agent possesses both serotonin reuptake inhibitory activity and norepinephrine reuptake inhibitory activity or is a reversible inhibitor of monoamine oxidase A (RIMA), comprising administering to the patient an effective amount of the first therapeutic agent and the second therapeutic agent. In some embodiments, at least one side effect that is reduced is daytime sedation, cognitive impairment, iatrogenic weight gain, nausea or emesis. In some embodiments, the first therapeutic agent comprises one or members of the group consisting of mirtazapine, mirtazapine enriched in either the R- or S-enantiomer, setiptiline or a combination thereof. In some embodiments, the first therapeutic agent comprises mirtazapine. In some embodiments, the first therapeutic agent comprises setiptiline. In some embodiments, when the second therapeutic agent possesses serotonin reuptake inhibitory activity and norepinephrine reuptake inhibitory activity; and the ratio of serotonin reuptake inhibitory activity to norepinephrine reuptake inhibitory activity is in the range of about 1:50 to 50:1, about 1:20 to 20:1 or about 1:10 to 10:1. In some embodiments, the second therapeutic agent comprises a serotonin norepinephrine reuptake inhibitor (SNRI). In some embodiments, the SNRI is selected from the group consisting of duloxetine, venlafaxine, desvenlafaxine, indeloxazine and pharmaceutically acceptable salts, derivatives and combinations thereof. In some embodiments, the second therapeutic agent comprises a norepinephrine serotonin reuptake inhibitor (NSRI). In some embodiments, the NSRI is selected from the group consisting of milnacipran, derivatives of milnacipran, bicifadine, and pharmaceutically acceptable salts, derivatives and combinations thereof. In some embodiments, the second therapeutic agent comprises a reversible inhibitor of monoamine oxidase A (RIMA). In some embodiments, the second therapeutic agent comprises a RIMA selected from the group consisting of brofaromine, moclobemide, and pharmaceutically acceptable salts, derivatives and combinations thereof. In some embodiments, the first and second therapeutic agents are administered as a unit dose. In some embodiments, the unit dose provides immediate release of at least a portion of the first therapeutic agent. In some embodiments, the unit dose provides immediate release of substantially all of the first therapeutic agent. In some embodiments, the unit dose provides delayed release of at least a portion of the second therapeutic agent. In some embodiments, the unit dose provides delayed release of substantially all of the second therapeutic agent. In some embodiments, the unit dose is administered to the patient within about 4 hours before bed, about 2 hours before bed, about 1 hour before bed or substantially immediately before bed. In some embodiments, the unit dose provides immediate release of at least a portion of the second therapeutic agent. In some embodiments, the unit dose provides immediate release of substantially all of the second therapeutic agent. In some embodiments, the unit dose provides delayed release of at least a portion of the first therapeutic agent. In some embodiments, the unit dose provides delayed release of substantially all of the first therapeutic agent. In some embodiments, the unit dose is administered to the patient within about 4 hours after waking, within 2 hours after waking, before, with or after a meal. In some embodiments, the first therapeutic agent is administered before bed and the second therapeutic agent is administered after waking. In some embodiments, the first therapeutic agent is administered within about 4 hours before bed, within about 2 hours before bed, within about 1 hour before bed or substantially immediately before bed. In some embodiments, the second therapeutic agent is administered within about 4 hours of waking, within about 2 hours after waking, within about 1 hour after waking, before, with or after a meal. In some embodiments, at least one side effect that is reduced is daytime sedation, cognitive impairment, iatrogenic weight gain, nausea or emesis. In some embodiments, the method provides reduction in two or more side effects selected from the group consisting of daytime sedation, cognitive impairment, iatrogenic weight gain, nausea and emesis. In some embodiments, the disorder is selected from the group consisting of depression, schizophrenia, anxiety disorders, affective disorders, sleep-related breathing disorders, insomnia, migraine headache, chronic tension-type headache, hot flashes, chronic lower back pain, neuropathic pain (e.g. from diabetic peripheral neuropathy) and functional somatic syndromes. In some embodiments, the disorder is an anxiety disorder selected from the group consisting of generalized anxiety disorder, panic disorder, phobias, and post-traumatic stress disorder. In some embodiments, the disorder is a sleep-related breathing disorder selected from the group consisting of sleep apnea, sleep hypopnea, upper airway resistance syndrome, and snoring. In some embodiments, the disorder is a functional somatic syndrome selected from the group consisting of fibromyalgia syndrome, chronic fatigue syndrome, and irritable bowel syndrome.

In some embodiments, the method of paragraph [0023] is carried out using a combination of mirtazapine (or a pharmaceutically acceptable stereoisomer or salt thereof) and a NSRI. In particular embodiments, the method of paragraph [0023] is carried out using a combination of mirtazapine or a pharmaceutically acceptable stereoisomer or salt thereof and milnacipran, or a pharmaceutically acceptable stereoisomer or salt thereof, or a derivative of milnacipran, or a pharmaceutically acceptable stereoisomer or salt thereof. In some more particular embodiments, the method of paragraph [0023] is carried out using a combination of mirtazapine or a pharmaceutically acceptable stereoisomer or salt thereof, and milnacipran, or a pharmaceutically acceptable stereoisomer or salt thereof.

In some embodiments, the present disclosure provides a formulation comprising an effective amount of a combination of a first therapeutic agent and a second therapeutic agent, wherein the first therapeutic agent is a 5HT₂/5HT₃ antagonist/alpha-2 antagonist and the second therapeutic agent possesses both serotonin reuptake inhibitory activity and norepinephrine reuptake inhibitory activity or is a reversible inhibitor of monoamine oxidase A (RIMA). In some embodiments, the first therapeutic agent comprises one or members of the group consisting of mirtazapine, mirtazapine enriched in either the R- or S-enantiomer, setiptiline or a combination thereof. In some embodiments, the first therapeutic agent comprises mirtazapine. In some embodiments, the first therapeutic agent comprises setiptiline. In some embodiments, the second therapeutic agent possesses serotonin reuptake inhibitory activity and norepinephrine reuptake inhibitory activity; and the ratio of serotonin reuptake inhibitory activity to norepinephrine reuptake inhibitory activity is in the range of about 1:50 to 50:1, about 1:20 to 20:1 or about 1:10 to 10:1. In some embodiments, the second therapeutic agent comprises a SNRI, NSRI or RIMA (SNRI). In some embodiments, the SNRI is selected from the group consisting of duloxetine, venlafaxine, desvenlafaxine, indeloxazine and pharmaceutically acceptable salts, derivatives and combinations thereof. In some embodiments, the second therapeutic agent comprises a NSRI. In some embodiments, the NSRI is selected from the group consisting of milnacipran, derivatives of milnacipran, bicifadine, and pharmaceutically acceptable salts, derivatives and combinations thereof. In some embodiments, the second therapeutic agent comprises a reversible inhibitor of monoamine oxidase A (RIMA). In some embodiments, the RIMA is selected from the group consisting of brofaromine, moclobemide, and pharmaceutically acceptable salts, derivatives and combinations thereof. In some embodiments, the first and second therapeutic agents are administered in a unit dose. In some embodiments, the unit dose provides immediate release of at least a portion of the first therapeutic agent. In some embodiments, the unit dose provides immediate release of substantially all of the first therapeutic agent. In some embodiments, the unit dose provides delayed release of at least a portion of the second therapeutic agent. In some embodiments, the unit dose provides delayed release of substantially all of the second therapeutic agent. In some embodiments, the unit dose is adapted to be administered to the patient within about 4 hours before bed, about 2 hours before bed, within about 1 hour of bed or substantially immediately before bed. In some embodiments, the unit dose provides immediate release of at least a portion of the second therapeutic agent. In some embodiments, the unit dose provides immediate release of substantially all of the second therapeutic agent. In some embodiments, the unit dose provides delayed release of at least a portion of the first therapeutic agent. In some embodiments, the unit dose provides delayed release of substantially all of the first therapeutic agent. In some embodiments, the unit dose is adapted to be administered to the patient within about 4 hours of waking, within about 2 hours of waking, within about 1 hour of waking, before, with or after a meal.

In some embodiments, the formulation of paragraph [0025] comprises a combination of mirtazapine (or a pharmaceutically acceptable stereoisomer or salt thereof) and a NSRI. In particular embodiments, the formulation of paragraph [0025] includes a combination of mirtazapine or a pharmaceutically acceptable stereoisomer or salt thereof and milnacipran, or a pharmaceutically acceptable stereoisomer or salt thereof, or a derivative of milnacipran, or a pharmaceutically acceptable stereoisomer or salt thereof. In some more particular embodiments, the formulation of paragraph [0025] contains a combination of mirtazapine or a pharmaceutically acceptable stereoisomer or salt thereof, and milnacipran, or a pharmaceutically acceptable stereoisomer or salt thereof. In some embodiments, the formulation of paragraph [0025] contains a combination of mirtazapine or a pharmaceutically acceptable stereoisomer or salt thereof, and milnacipran, or a pharmaceutically acceptable stereoisomer or salt thereof; and mirtazapine and milnacipran are the sole active ingredients in the formulation.

In some embodiments, the present disclosure provide a method of treating a disorder treatable by administration of a first therapeutic agent having 5HT2/5HT3 antagonist and alpha-2 antagonist activity, a second therapeutic agent, or both, wherein the second therapeutic agent possesses both serotonin reuptake inhibitory activity and norepinephrine reuptake inhibitory activity, the method comprising administering the first therapeutic agent to the patient, and within about 18 hours of administering the first therapeutic agent, administering the second therapeutic agent, wherein combined administration of the first therapeutic agent and the second therapeutic agent is effective to treat at least one disorder, wherein a reduction in at least one side effect associated with the first therapeutic agent, the second therapeutic agent, or both is obtained, and wherein at least one such side effect is selected from the group consisting of daytime sedation, nausea, emesis, weight gain and cognitive impairment. In some embodiments, the first therapeutic agent comprises one or members of the group consisting of mirtazapine, mirtazapine enriched in either the R- or S-enantiomer, setiptiline or a combination thereof. In some embodiments, the first therapeutic agent mirtazapine. In some embodiments, the first therapeutic agent comprises setiptiline. In some embodiments, the second therapeutic agent possesses serotonin reuptake inhibitory activity and norepinephrine reuptake inhibitory activity; and the ratio of serotonin reuptake inhibitory activity to norepinephrine reuptake inhibitory activity is in the range of about 1:50 to 50:1, about 1:20 to 20:1 or about 1:10 to 10:1. In some embodiments, the second therapeutic agent comprises a serotonin norepinephrine reuptake inhibitor (SNRI). In some embodiments, the SNRI is selected from the group consisting of duloxetine, venlafaxine, desvenlafaxine, indeloxazine and pharmaceutically acceptable salts, derivatives and combinations thereof. In some embodiments, the second therapeutic agent comprises a norepinephrine serotonin reuptake inhibitor (NSRI). In some embodiments, the NSRI is selected from the group consisting of milnacipran, derivatives of milnacipran, bicifadine, and pharmaceutically acceptable salts, derivatives and combinations thereof. In some embodiments, the second therapeutic agent comprises a reversible inhibitor of monoamine oxidase A (RIMA). In some embodiments, the RIMA is selected from the group consisting of brofaromine, moclobemide, and pharmaceutically acceptable salts, derivatives and combinations thereof. In some embodiments, the first and second therapeutic agents are administered in a unit dose. In some embodiments, the unit dose provides immediate release of at least a portion of the first therapeutic agent. In some embodiments, the unit dose provides immediate release of substantially all of the first therapeutic agent. In some embodiments, the unit dose provides delayed release of at least a portion of the second therapeutic agent. In some embodiments, the unit dose provides delayed release of substantially all of the second therapeutic agent. In some embodiments, the unit dose is administered to the patient within about 4 hours before bed, within about 2 hours of bed, within about 1 hour of bed or substantially immediately before bed. In some embodiments, the unit dose provides immediate release of at least a portion of the second therapeutic agent. In some embodiments, the unit dose provides immediate release of substantially all of the second therapeutic agent. In some embodiments, the unit dose provides delayed release of at least a portion of the first therapeutic agent. In some embodiments, the unit dose provides delayed release of substantially all of the first therapeutic agent. In some embodiments, the unit dose is administered to the patient within about 4 hours after waking, within about 2 hours after waking, within about 1 hour after waking, before, with or after a meal. In some embodiments, the first therapeutic agent is administered before bed and the second therapeutic agent is administered after waking. In some embodiments, the first therapeutic agent is administered within about 4 hours before bed, within about 2 hours before bed, within about 1 hour before bed or substantially immediately before bed. In some embodiments, the second therapeutic agent is administered within about 4 hours of waking, within about 2 hours of waking, within about 1 hour of waking, before, with or after a meal. In some embodiments, the method provides a reduction in two or more side effects selected from the group consisting of daytime sedation, cognitive impairment, iatrogenic weight gain, nausea and emesis. In some embodiments, the disorder is selected from the group consisting of depression, schizophrenia, anxiety disorders, affective disorders, sleep-related breathing disorders, insomnia, migraine headache, chronic tension-type headache, hot flashes, chronic lower back pain, neuropathic pain (e.g. from diabetic peripheral neuropathy) and functional somatic syndromes. In some embodiments, the disorder is an anxiety disorder selected from the group consisting of generalized anxiety disorder, panic disorder, phobias, and post-traumatic stress disorder. In some embodiments, the disorder is a sleep-related breathing disorder selected from the group consisting of sleep apnea, sleep hypopnea, upper airway resistance syndrome, and snoring. In some embodiments, the disorder is a functional somatic syndrome selected from the group consisting of fibromyalgia syndrome, chronic fatigue syndrome, and irritable bowel syndrome.

In some embodiments, the method of paragraph [0027] is carried out using a combination of mirtazapine (or a pharmaceutically acceptable stereoisomer or salt thereof) and a NSRI. In particular embodiments, the method of paragraph [0027] is carried out using a combination of mirtazapine or a pharmaceutically acceptable stereoisomer or salt thereof and milnacipran, or a pharmaceutically acceptable stereoisomer or salt thereof, or a derivative of milnacipran, or a pharmaceutically acceptable stereoisomer or salt thereof. In some more particular embodiments, the method of paragraph [0027] is carried out using a combination of mirtazapine or a pharmaceutically acceptable stereoisomer or salt thereof, and milnacipran, or a pharmaceutically acceptable stereoisomer or salt thereof.

In some embodiments, the disclosure herein provides a kit comprising a first therapeutic agent, a second therapeutic agent and instructions for administering the first therapeutic agent before bed and the second therapeutic agent after waking, wherein the first therapeutic agent comprises a 5HT₂/5HT₃ antagonist/alpha-2 antagonist and the second therapeutic agent possesses both serotonin reuptake inhibitory activity and norepinephrine reuptake inhibitory activity or is a reversible inhibitor of monoamine oxidase A (RIMA). In some embodiments, the first therapeutic agent comprises one or members of the group consisting of mirtazapine, mirtazapine enriched in either the R- or S-enantiomer, setiptiline or a combination thereof. In some embodiments, the 5HT₂/5HT₃ antagonist/alpha-2 antagonist comprises mirtazapine. In some embodiments, the 5HT₂/5HT₃ antagonist/alpha-2 antagonist comprises setiptiline. In some embodiments, the second therapeutic agent possesses serotonin reuptake inhibitory activity and norepinephrine reuptake inhibitory activity; and the ratio of serotonin reuptake inhibitory activity to norepinephrine reuptake inhibitory activity is in the range of about 1:50 to 50:1, about 1:20 to 20:1 or about 1:10 to 10:1. In some embodiments, the second therapeutic agent comprises a serotonin norepinephrine reuptake inhibitor (SNRI). In some embodiments, the SNRI is selected from the group consisting of duloxetine, venlafaxine, desvenlafaxine, indeloxazine and pharmaceutically acceptable salts, derivatives and combinations thereof. In some embodiments, the second therapeutic agent comprises a norepinephrine serotonin reuptake inhibitor (NSRI). In some embodiments, the NSRI is selected from the group consisting of milnacipran, derivatives of milnacipran, bicifadine, and pharmaceutically acceptable salts, derivatives and combinations thereof. In some embodiments, the second therapeutic agent comprises a reversible inhibitor of monoamine oxidase A (RIMA). In some embodiments, the second therapeutic agent comprises a RIMA selected from the group consisting of brofaromine, moclobemide, and pharmaceutically acceptable salts, derivatives and combinations thereof. In some embodiments, the kit comprises instructions to administer the first therapeutic agent within about 4 hours before bed, within about 2 hours before bed, within about 1 hour before or substantially immediately before bed. In some embodiments, the kit comprises instructions to administer the second therapeutic agent within about 4 hours of waking, within about 2 hours after waking, within about 1 hour after waking, before, with or after a meal.

In some embodiments, the kit of paragraph [0029] contains a combination of mirtazapine (or a pharmaceutically acceptable stereoisomer or salt thereof) and a NSRI. In particular embodiments, the kit of paragraph [0029] is includes a combination of mirtazapine or a pharmaceutically acceptable stereoisomer or salt thereof and milnacipran, or a pharmaceutically acceptable stereoisomer or salt thereof, or a derivative of milnacipran, or a pharmaceutically acceptable stereoisomer or salt thereof. In some more particular embodiments, the kit of paragraph [0029] contains a combination of mirtazapine or a pharmaceutically acceptable stereoisomer or salt thereof, and milnacipran, or a pharmaceutically acceptable stereoisomer or salt thereof. In some embodiments, the kit of paragraph [0029] contains a combination of mirtazapine or a pharmaceutically acceptable stereoisomer or salt thereof, and milnacipran, or a pharmaceutically acceptable stereoisomer or salt thereof; and mirtazapine and milnacipran are the sole active agents in the kit.

Some embodiments described herein provide a unit dosage form containing a synergistic combination of a first therapeutic agent and a second therapeutic agent, wherein the first therapeutic agent comprises a 5HT₂/5HT₃ antagonist/alpha-2 antagonist and the second therapeutic agent possesses both serotonin reuptake inhibitory activity and norepinephrine reuptake inhibitory activity norepinephrine or is a reversible inhibitor of monoamine oxidase A (RIMA). In some embodiments, the unit dosage provides effective treatment of at least one disorder selected from the group consisting of depression, schizophrenia, anxiety disorders, affective disorders, sleep-related breathing disorders, insomnia, migraine headache, chronic tension-type headache, hot flashes, chronic lower back pain, neuropathic pain (e.g. from diabetic peripheral neuropathy) and functional somatic syndromes. In some embodiments, the disorder is an anxiety disorder selected from the group consisting of generalized anxiety disorder, panic disorder, phobias, and post-traumatic stress disorder. In some embodiments, the disorder is a sleep-related breathing disorder selected from the group consisting of sleep apnea, sleep hypopnea, upper airway resistance syndrome, and snoring. In some embodiments, the disorder is a functional somatic syndrome selected from the group consisting of fibromyalgia syndrome, chronic fatigue syndrome, and irritable bowel syndrome. In some embodiments, the first therapeutic agent comprises one or members of the group consisting of mirtazapine, mirtazapine enriched in either the R- or S-enantiomer, setiptiline, a pharmaceutically acceptable salt of mirtazapine (or a stereoisomer thereof) and a pharmaceutically setiptiline (or a stereoisomer thereof). In some embodiments, the first therapeutic agent comprises mirtazapine. In some embodiments, the first therapeutic agent comprises setiptiline. In some embodiments, the second therapeutic agent possesses serotonin reuptake inhibitory activity and norepinephrine reuptake inhibitory activity; and the ratio of serotonin reuptake inhibitory activity to norepinephrine reuptake inhibitory activity is in the range of about 1:50 to 50:1, about 1:20 to 20:1 or about 1:10 to 10:1. In some embodiments, the second therapeutic agent comprises a SNRI. In some embodiments, the SNRI is a member of the group consisting of duloxetine, venlafaxine, desvenlafaxine, indeloxazine and pharmaceutically acceptable salts, derivatives and combinations thereof. In some embodiments, the second therapeutic agent comprises a NSRI. In some embodiments, the NSRI is selected from the group consisting of milnacipran, derivatives of milnacipran, bicifadine, and pharmaceutically acceptable salts, derivatives and combinations thereof. In some embodiments, the second therapeutic agent comprises a reversible inhibitor of monoamine oxidase A (RIMA). In some embodiments, the second therapeutic agent comprises a RIMA selected from the group consisting of brofaromine, moclobemide, and pharmaceutically acceptable salts, derivatives and combinations thereof. In some embodiments, the unit dose provides immediate release of at least a portion of the first therapeutic agent. In some embodiments, the unit dose provides immediate release of substantially all of the first therapeutic agent. In some embodiments, the unit dose provides delayed release of at least a portion of the second therapeutic agent. In some embodiments, the unit dose provides delayed release of substantially all of the second therapeutic agent. In some embodiments, the unit dose is adapted to be administered to the patient within about 4 hours before bed, within about 2 hours before bed, within about 1 hour before bed or substantially immediately before bed. In some embodiments, the unit dose provides immediate release of at least a portion of the second therapeutic agent. In some embodiments, the unit dose provides immediate release of substantially all of the second therapeutic agent. In some embodiments, the unit dose provides delayed release of at least a portion of the first therapeutic agent. In some embodiments, the unit dose provides delayed release of substantially all of the first therapeutic agent. In some embodiments, the unit dose is administered to the patient within about 4 hours after waking, within about 2 hours after waking, within about 1 hour after waking, before, with or after a meal. In some embodiments, the unit dose comprises about 0.5-45 mg of mirtazapine and about 10 to about 400 mg of milnacipran. In some embodiments, the unit dose comprises about 0.5 to about 5 mg of mirtazapine and about 20 to about 200 of milnacipran. In some embodiments, the unit dose comprises about 0.5-45 mg of mirtazapine and about 5 to 100 mg of duloxetine or a pharmaceutically acceptable salt or stereoisomer thereof. In some embodiments, the unit dose comprises about 0.5 to about 5 mg of mirtazapine and about 10 to about 80 mg of duloxetine or a pharmaceutically acceptable salt or stereoisomers thereof. In some embodiments, the unit dose contains less than 100% of the average effective dose of 5HT₂/5HT₃ antagonist/alpha-2 antagonist and less than 100% of the average effective dose of SNRI, NSRI or RIMA. In some embodiments, the unit dose contains less than about 75% of the average effective dose of 5HT₂/5HT₃ antagonist/alpha-2 antagonist and less than 75% of the average effective dose of the second therapeutic agent. In some embodiments, the unit dose contains only about 0.5 to 161% of the average effective dose of 5HT₂/5HT₃ antagonist/alpha-2 antagonist and about 0.5 to 161% of the average effective dose the second therapeutic agent.

In some embodiments, the unit dosage form of paragraph [0031] contains a combination of mirtazapine (or a pharmaceutically acceptable stereoisomer or salt thereof) and a NSRI. In particular embodiments, the unit dosage form of paragraph [0031] includes a combination of mirtazapine or a pharmaceutically acceptable stereoisomer or salt thereof and milnacipran, or a pharmaceutically acceptable stereoisomer or salt thereof, or a derivative of milnacipran, or a pharmaceutically acceptable stereoisomer or salt thereof. In some more particular embodiments, the unit dosage form of paragraph [0031] comprises out using a combination of mirtazapine or a pharmaceutically acceptable stereoisomer or salt thereof, and milnacipran, or a pharmaceutically acceptable stereoisomer or salt thereof. In some more particular embodiments, the unit dosage form of paragraph [0031] comprises out using a combination of mirtazapine or a pharmaceutically acceptable stereoisomer or salt thereof, and milnacipran, or a pharmaceutically acceptable stereoisomer or salt thereof; and mirtazapine and milnacipran are the sole active agents in the combination.

### Active Ingredients, in General

The person of skill in the art will recognize that the compositions described herein contain active ingredients, which may take on multiple solid physical forms, such as pharmaceutically acceptable salts, enantiomers, stereoisomers, diastereomers, enantiomerically enriched mixtures, polymorphs and amorphous solids. Where a particular active pharmaceutical ingredient (“API”) is referred to herein without further qualification, it is to be understood that such reference encompasses any of the possible physical forms of that API, including, if applicable: (1) enantiomers, stereoisomers, diastereomers, enantiomerically enriched mixtures of the API; (2) pharmaceutically acceptable salts of the API or any applicable enantiomers, stereoisomers, diastereomers, enantiomerically enriched mixtures of the API; (3) any applicable polymorphs of the API or any applicable enantiomers, stereoisomers, diastereomers, enantiomerically enriched mixtures of the API or pharmaceutically acceptable salts thereof; or (4) amorphous solids of: (a) the API, (b) pharmaceutically acceptable salts of the API, or (c) any applicable enantiomers, stereoisomers, diastereomers, enantiomerically enriched mixtures of the API or pharmaceutically acceptable salts thereof. In this regard, the term “derivative” of an API may be used herein to encompass a pharmaceutically acceptable salt, enantiomer, diastereomer, stereoisomer, enantiomerically enriched, amorphous solid or polymorphic form of the API. Where a more specific physical form is intended, such as a specific salt or enantiomerically enriched mixture, this will be called out in more specific terms. For example, as used herein “mirtazapine” refers to mirtazapine in any of its physical forms, such free base, a pharmaceutically acceptable salt, enantiomerically pure R-mirtazapine or S-mirtazapine, a pharmaceutically acceptable salt of R-mirtazapine or S-mirtazapine, etc.

Drugs with 5HT₂/5HT₃ Serotonin Receptor Antagonist and Alpha-2 Adrenergic Receptor Antagonist Activity

Useful drugs include compounds that act as antagonists at both the 5HT₂ and 5HT₃ serotonin receptors and at alpha-2 adrenergic receptors (5HT₂/5HT₃ antagonist/alpha-2 antagonists). In some embodiments provided herein, such compounds are mirtazapine (1,2,3,4,10,14b-hexahydro-2-methylpyrazino[2,1-a]pyrido[2,3-c]benzazepine), setiptiline (1,2,3,4-tetrahydro-2-methyl-9H-dibenzo[3,4:6,7]cyclohepta[1,2-C]pyridine maleate) in the form of their free bases or pharmaceutically acceptable salts.

### Mirtazapine

Mirtazapine is currently approved in multiple countries for the treatment of depression; the first approval occurred in 1994. Mirtazapine's chemical name is 1,2,3,4,10,14b-hexahydro-2-methylpyrazino [2,1-a]pyrido[2,3-c]benzazepine. The chemical structure is as follows:

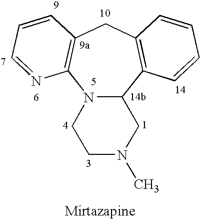

As is clear from the structure, mirtazapine is a chiral compound, and only the racemate has been commercialized to date. Nonetheless, the activity of mirtazapine may reside primarily in one or the other of the R- and S-enantiomers of mirtazapine. (Some references, such as WO/2007/063042, incorporated herein in its entirety, suggest that R-enantiomer is more active than the S-enantiomer). Hence, reference to mirtazapine, unless otherwise modified herein, embraces the racemate, each of the enantiomers in a purified state and mixtures of the enantiomers in all possible ratios, as well as pharmaceutically acceptable salts and polymorphs thereof. In some embodiments, the pharmaceutically active ingredient designated as mirtazapine is a 50:50 ratio of the R- and S-enantiomers of 1,2,3,4,10,14b-hexahydro-2-methylpyrazino [2,1-a]pyrido[2,3-c]benzazepine. In some embodiments, the term mirtazapine includes mixture or enantiomers of mirtazapine having a ratio of R- to S-enantiomer (mass/mass) in the range of about 50:50 to about 100:0, about 50:50 to about 60:40, about 50:50 to about 70:30, about 50:50 to about 80:20, about 50:50 to about 90:10, about 50:50 to about 95:5, about 50:50 to about 97.5:2.5, about 50:50 to about 99:1, about 50:50 to about 99.5:0.5, about 50:50 to about 99.9:0.1, about 60:40 to about 100:0, about 60:40 to about 70:30, about 60:40 to about 80:20, about 60:40 to about 90:10, about 60:40 to about 95:5, about 60:40 to about 97.5:2.5, about 60:40 to about 99:1, about 60:40 to about 99.5:0.5, about 60:40 to about 99.9:0.1, about 60:40 to about 100:0, about 70:30 to about 80:20, about 70:30 to about 90:10, about 70:30 to about 95:5, about 70:30 to about 97.5:2.5, about 70:30 to about 99:1, about 70:30 to about 99.5:0.5, about 70:30 to about 99.9:0.1, about 75:25 to about 80:20, about 75:25 to about 90:10, about 75:25 to about 95:5, about 75:25 to about 97.5:2.5, about 75:25 to about 99:1, about 75:25 to about 99.5:0.5, about 75:25 to about 99.9:0.1. In other embodiments, the ratio of the S- to the R-enantiomer may be in the range of 50:50 to about 100:0, about 50:50 to about 60:40, about 50:50 to about 70:30, about 50:50 to about 80:20, about 50:50 to about 90:10, about 50:50 to about 95:5, about 50:50 to about 97.5:2.5, about 50:50 to about 99:1, about 50:50 to about 99.5:0.5, about 50:50 to about 99.9:0.1, about 60:40 to about 100:0, about 60:40 to about 70:30, about 60:40 to about 80:20, about 60:40 to about 90:10, about 60:40 to about 95:5, about 60:40 to about 97.5:2.5, about 60:40 to about 99:1, about 60:40 to about 99.5:0.5, about 60:40 to about 99.9:0.1, about 60:40 to about 100:0, about 70:30 to about 80:20, about 70:30 to about 90:10, about 70:30 to about 95:5, about 70:30 to about 97.5:2.5, about 70:30 to about 99:1, about 70:30 to about 99.5:0.5, about 70:30 to about 99.9:0.1, about 75:25 to about 80:20, about 75:25 to about 90:10, about 75:25 to about 95:5, about 75:25 to about 97.5:2.5, about 75:25 to about 99:1, about 75:25 to about 99.5:0.5, about 75:25 to about 99.9:0.1. The mechanism by which mirtazapine exerts its antidepressant effects is not fully understood, a situation that is consistent with other drugs approved for use for depression. Pharmacologically, mirtazapine enhances central noradrenergic and serotonergic activity. However, the agent has minimal effects upon peripheral serotonin levels, thus minimizing the chance for serotonin syndrome when used in combination with SSRI or TCA antidepressants. Studies have shown that mirtazapine acts as an antagonist at central presynaptic α₂ adrenergic inhibitory autoreceptors and heteroreceptors, an action that is postulated to result in an increase in central noradrenergic and serotonergic activity. Mirtazapine is a potent antagonist of 5-HT₂ and 5-HT₃ receptors, but lacks significant affinity for the 5-HT_{1A} and 5-HT_{1B} receptors. Mirtazapine is a potent antagonist of histamine (H₁) receptors, a property that may explain its prominent sedative effects. Mirtazapine may also reduce nausea by specific inhibition of the serotonin 5HT₃ receptor. Mirtazapine is a moderate peripheral α₁ adrenergic antagonist, a property that may explain the occasional orthostatic hypotension reported in association with its use. Mirtazapine is a moderate antagonist at muscarinic receptors, a property that may explain the relatively low incidence of anti-cholinergic side effects, including cognitive impairment, associated with its use.

### Setiptiline

Setiptiline ((1,2,3,4-tetrahydro-2-methyl-9H-dibenzo[3,4:6,7]cyclohepta[1,2-C]pyridine maleate), also variously identified as 13b,4a-carba-mianserin; MO 8282; MO-8282; ORG 8282; and ORG-8282) is a drug having antagonist activity toward the central 5HT₂, 5HT₃ and α₂ receptors and possesses indications and pharmacology that are very similar to those of mirtazapine. Considering that the mirtazapine and setiptiline have similar microbiological activity, it is likely that setiptiline will have a side effect profile similar to that of mirtazapine. The structure of setiptiline appears below.

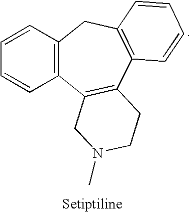

Drugs with Serotonin Norepinephrine Reuptake Inhibiting Activity (SNRIs)

Serotonin norepinephrine reuptake inhibitors (SNRIs) are therapeutic agents that increase the intrasynaptic concentration of both serotonin and norepinephrine by inhibiting their reuptake. With SNRIs, the serotonergic effect is greater than the noradrenergic effect. The class of SNRIs includes duloxetine, venlafaxine, desvenlafaxine and derivatives, stereoisomers and pharmaceutically acceptable salts thereof.

Duloxetine ((+)-(S)—N-methyl-3-(1-naphthyloxy)-3-(thiophen-2-yl)-propan-1-amine) is a SNRI marketed as the hydrochloride (HCl) salt under the brand name Cymbalta® by Eli Lilly. Duloxetine is indicated for treatment of major depressive disorder, generalized anxiety disorder, pain related to diabetic neuropathy and stress urinary incontinence. Among the more common side effects of duloxetine are nausea, somnolence, insomnia and dizziness. Other infrequent, but serious, side effects include sexual side effects.

Venlafaxine (1-[2-dimethylamino-1-(4-methoxyphenyl)-ethyl]cyclohexan-1-ol) is marketed under the brand name Effexor® by Wyeth Pharmaceuticals. Venlafaxine is a potent inhibitor of monoamine (serotonin and norepinephrine) reuptake, and to a lesser extent of dopamine reuptake. Although originally developed as an antidepressant (U.S. Pat. No. 4,535,186), venlafaxine is also indicated for the treatment of generalized anxiety disorder (U.S. Pat. No. 5,916,923), social anxiety disorder, and panic disorder. It is also believed that venlafaxine provides relief for diabetic neuropathy and migraine. Prescribed dosages are typically in the range of 75 to 225 mg per day, but higher dosages up to 450 mg are sometimes used for the treatment of severe or treatment-resistant depression. Some side effects of venlafaxine include nausea, emesis, insomnia and fatigue. These side effects may be seen as treatment limiting or impairing in some instances.

Desvenlafaxine (4-[2-dimethylamino-1-(1-hydroxycyclohexyl)ethyl]phenol) is an isolated active metabolite of venlafaxine and is expected to be marketed in the United States as the succinate under the brand name Pristiq® by Wyeth Pharmaceuticals upon final approval. The indications for desvenlafaxine are expected to be similar to those of venlafaxine; however there is evidence that desvenlafaxine may be useful in the treatment of climacteric effects of menopause and perimenopause. The side effects of desvenlafaxine are similar to those of venlafaxine and include nausea, insomnia and somnolence. As in the case of venlafaxine, one or more of these side effects could inhibit or limit treatment or interfere with patient compliance.

Indeloxazine (2-(7-indenyloxymethyl)morpholine) is currently marketed as the hydrochloride salt for the treatment of post-stroke depression, emotional disturbance and reduced volition in Japan and South Korea. (Yamaguchi et al., “Neurochemical and behavioral characterization of potential antidepressant properties of indeloxazine hydrochloride,” Neuropharmacology 37 (1998), 1169-1176. Indeloxazine hydrochloride is an inhibitor of serotonin reuptake and of norepinephrine reuptake. Id. The manufacture and antidepressant activity of indeloxazine and related compounds are described in U.S. Pat. No. 4,109,088, which is incorporated herein by reference in its entirety. In this context, a “derivative of indeloxazine,” refers to one or more of the related compounds described specifically or generically in the '088 patent. Indeloxazine has one stereogenic center at the 2-position. Thus, certain embodiments described herein provide compositions comprising racemic indeloxazine, R-indeloxazine, or S-indeloxazine, or a pharmaceutically acceptable salt thereof. Thus, as used herein, “indeloxazine” refers to indeloxazine in all its forms, e.g. racemic mixture, R-indeloxazine and S-indeloxazine, unless otherwise qualified. In some embodiments, the dosage for Indeloxazine is about 5 to about 1000 mg/day, about 10 to about 500 mg/day, or about 50 to about 300 mg/day.

Drugs with Norepinephrine Serotonin Reuptake Inhibiting Activity (NSRIs)

A norepinephrine serotonin reuptake inhibitors (NSRI) is a composition that inhibits the reuptake of both norepinephrine and serotonin, thereby increasing the concentration of intrasynaptic serotonin and norepinephrine. What distinguishes NSRI from SNRI compounds is that the former have norepinephrine reuptake inhibitory activity that is at least as great as, if not greater than, their serotonin reuptake activity. Exemplary SNRI compounds according to the present invention include, but are not necessarily limited to, duloxetine, venlafaxine, desvenlafaxine and pharmaceutically acceptable salts thereof.

Milnacipran (cis-(±)-2-(aminomethyl)-N,N-diethyl-1-phenylcyclopropanecarboxamide; also known as midalcipran) is classified as a Norepinephrine Serotonin Reuptake Inhibitor (NSRI) because it has nearly equal potency for inhibiting the reuptake of both noradrenaline and serotonin, in vivo, although it has a ratio of norepinephrine reuptake inhibitory activity to serotonin reuptake inhibitory activity of about 3:1 in vitro. Originally developed for treatment of depression, milnacipran has been shown in phase III clinical trials to be effective in the treatment of fibromyalgia, a chronic syndrome characterized by diffuse or specific muscle, joint or bone fatigue and/or pain. It is believed that milnacipran's inhibition of the reuptake of noradrenaline is responsible for its analgesic activity. The manufacture of milnacipran is described in U.S. Pat. No. 4,478,836, which is incorporated herein by reference in its entirety.

Milnacipran has two stereogenic centers, which would theoretically give rise to four stereoisomers—two enantiomeric pairs. However, manufacture of milnacipran according to the method outlined in U.S. Pat. No. 4,478,836 results in a racemic mixture of the enantiomers having a substitution pattern on the cyclopropane ring in the cis-geometry. Thus, milnacipran described in U.S. Pat. No. 4,478,836 is a racemic mixture of the 1R,2S- and 1S,2R-enantiomers, each of which has a cis-geometry with respect to the cyclopropane ring. Hence the chemical formula for currently commercially available milnacipran is: cis (±)-2-(aminomethyl)-N,N-diethyl-1-phenylcyclopropanecarboxamide. This cis-racemate of milnacipran is a 50:50 (mass/mass) mixture of the (1R,2S) and (1S,2R) enantiomers:

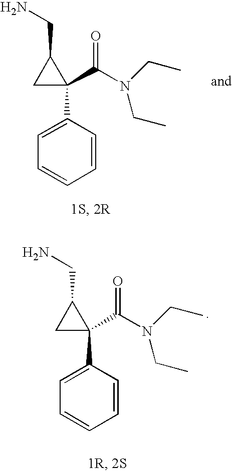

These two enantiomers can be separated and isolated using procedures described in the literature (Bonnaud et al., 1985, Journal of Chromatography, Vol. 318: 398 403; Shuto et al., Tetrahedron letters, 1996 Vol. 37:641 644; Grard et al., 2000, Electrophoresis 2000 21: 3028 3034; Doyle and Hu, 2001, Advanced Synthesis and Catalysis, Vol. 343: 299 302) as described in U.S. Pat. No. 7,074,833, which is incorporated herein by reference in its entirety. As described in U.S. Pat. No. 7,005,452, milnacipran that is enriched in the 1S,2R-enantiomer is dextrogyral, so the term dextrogyral milnacipran may be used herein to identify milnacipran that is enriched in the 1S,2R-enantiomer of milnacipran.

As used herein, unless otherwise qualified, the term milnacipran refers to the above-mentioned cis-racemate as well as dextrogyral milnacipran, i.e. mixtures of the 1S,2R-2S,1R-enantiomers in all possible ratios. In some embodiments, milnacipran may be the 50:50 mixture of the 1S,2R and 1R,2S enantiomers (cis-racemate) that is currently commercially available as milnacipran, or a pharmaceutically acceptable salt thereof. In other embodiments, milnacipran may be enriched in one or the other of the enantiomers, or a pharmaceutically acceptable salt thereof. In particular embodiments, the active pharmaceutical ingredient milnacipran may be enriched in the 1S,2R enantiomer. In some embodiments, the mass/mass ratio of the (1S,2R) enantiomer to the (1R,2S) enantiomer of milnacipran is greater than 1:1. In some embodiments, the mass/mass ratio of the (1S,2R) enantiomer to the (1R,2S) enantiomer is greater than or equal to about 55:45, greater than or equal to about 60:40, greater than or equal to about 65:35, greater than or equal to about 70:30, greater than or equal to about 75:25, greater than or equal to about 80:20, greater than or equal to about 82:18, greater than or equal to about 84:16, greater than or equal to about 86:14, greater than or equal to about 88:12, greater than or equal to about 90:10, greater than or equal to about 91:9, greater than or equal to about 92:8, greater than or equal to about 93:7, greater than or equal to about 94:6, greater than or equal to about 95:5, greater than or equal to about 96:4, greater than or equal to about 97:3, greater than or equal to about 98:2, greater than or equal to about 99:1, greater than or equal to about 99.5:0.5. In some embodiments, the mass/mass ratio of the 1S,2R enantiomer to the 1R,2S enantiomer is in a range of about 55:45 to about 95:5, in a range of about 55:45 to about 92.5:7.5, in a range of about 55:45 to about 90:10, in a range of about 60:40 to about 95:5, in a range of about 60:40 to about 92.5:7.5, in a range of about 60:40 to about 90:10, in a range of about 65:35 to about 95:5, in a range of about 65:35 to about 92.5:7.5, in a range of about 65:35 to about 90:10, in a range of about 60:30 to about 95:5, in a range of about 70:30 to about 92.5:7.5, in a range of about 70:30 to about 90:10. Where a specific enantiomer is intended herein, it will be recited specifically. Where a particular ratio of one enantiomer to another is intended, it will be recited specifically.

Bicifadine (1-(4-methylphenyl)-3-azabicyclo[3.1.0]hexane hydrochloride) is a NSRI under development as an analgesic for the treatment of acute and chronic pain. The synthesis and anxiolytic and analgesic properties of bicifadine were originally described in U.S. Pat. No. 4,231,935, incorporated herein by reference in its entirety, (where it is identified by its synonym 1-(p-tolyl)-3-azabicyclo[3.1.0]hexane hydrochloride) and polymorphs are described in U.S. Pat. No. 7,094,799, incorporated herein by reference in its entirety. The side effects of bicifadine include nausea, vomiting, restlessness, headache and dyspepsia.

### Reversible Inhibitors of Monoamine Oxidase A (RIMAs)

Reversible inhibitor of monoamine oxidase A (RIMA) are monoamine oxidase inhibitors (MAOIs) that specifically antagonize monoamine oxidase A. Because the B monoamine oxidase isoenzyme remains uninhibited, a patient receiving a RIMA can still metabolize tyramine, and is thus not required to follow a strict tyramine-free diet. Because RIMA drugs inhibit monoamine oxidase A reversibly, they tend to be better tolerated than most MAOI drugs. Brofaromine and moclobemide are two RIMAs that may be.

Brofaromine (4-(7-Bromo-5-methoxybenzofuran-2-yl)piperidine) is a RIMA drug originally described in U.S. Pat. No. 4,231,935 as an antidepressant. Brofaromine has also been tested for anxiolytic, anti-bulimic and anti-social phobic effects. Side effects of brofaromine include nausea, xerostomia and sleep disturbances.

Moclobemide (4-chloro-N-[2-(4-morpholinyl)ethyl]benzamide) is RIMA drug that was described in U.S. Pat. No. 4,210,754 and is categorized as an antidepressant. Moclobemide acts on epinephrine (adrenaline), norepinephrine (noradrenaline), serotonin, and dopamine reuptake transporters. Its pharmacodynamic action encompasses activation, elevation of mood, and improvement of symptoms like dysphoria, fatigue, and difficulties in concentration. The duration and quality of sleep may also be improved. In the treatment of depression the antidepressant effect often becomes evident in the first week of therapy (earlier as noted with TCAs/SSRIs). Side effects of moclobemide include dizziness, nausea and insomnia.

### Salts, Stereoisomers, Polymorphs and Derivatives

Although described above with reference specific to compounds, one can also utilize stereoisomers, polymorphs, metabolites, derivates and salts of the active compounds. Methods for synthesis of these compounds are known to those skilled in the art. Examples of pharmaceutically acceptable salts include, but are not limited to, mineral or organic acid salts of basic residues such as amines, and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Conventional non-toxic salts include those derived from inorganic acids such as hydrochloric, hydrobromic, sulfuric, sulfamic, phosphoric and nitric acid; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic and isethionic acids. The pharmaceutically acceptable salts can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed. (Mack Publishing Company, Easton, Pa., 19143, p. 1418). It is common practice in the art to prepare drug substances as their pharmaceutically acceptable salts in order to improve their physical properties, e.g. to render them more manageable in the formulation process. It is to be understood that, unless otherwise specified, reference to or mention of a particular substance (e.g. mirtazapine, milnacipran, duloxetine, setiptiline, bicifadine, venlafaxine, desvenlafaxine, brofaromine or moclobemide) generically includes acid or, where appropriate, base addition salts thereof. It is likewise to be understood that, unless otherwise specified, where reference to or mention of a substance relates to its properties or use as a drug, said reference or mention of the drug generically includes pharmaceutically acceptable salts of the drug. Where the free base (or acid) of a drug is intended, it is will be specifically and unambiguously stated, e.g. by referring to “the free base of drug A,” etc.

Stereoisomers are compounds made up of the same atoms having the same bond order but having different three-dimensional arrangements of atoms, which are not interchangeable. The three-dimensional structures are called configurations. Two kinds of stereoisomers that may be mentioned are enantiomers and diastereomers. Enantiomers are two stereoisomers which are non-superimposable mirror images of one another. This property of enantiomers is known as chirality. The terms “racemate”, “racemic mixture” or “racemic modification” refer to a mixture of equal parts of enantiomers. The term “chiral center” refers to a carbon atom to which four different groups are attached. Choice of the appropriate chiral column, eluent, and conditions necessary to effect separation of the pair of enantiomers is well known to one of ordinary skill in the art using standard techniques (see e.g. Jacques, J. et al., “Enantiomers, Racemates, and Resolutions”, John Wiley and Sons, Inc. 1981). Diastereomers are two stereoisomers which are not mirror images but are also not superimposable. Diastereoisomers have different physical properties and can be separated from one another easily by taking advantage of these differences. Unless otherwise specified herein, recitation of a substance without reference to its configuration means that the racemate as well as any and all isolated stereoisomers and enantiomers, as well as mixtures thereof, are included within the term. Where a particular stereoisomer, enantiomer or mixture of stereoisomers or enantiomers is intended, it will be stated specifically and unambiguously. Unless otherwise specified, reference to a particular active pharmaceutical ingredient is intended to include the currently commercially available form of the active pharmaceutical ingredient (if applicable), a racemate (if applicable), or a mixture of two or more stereoisomers in every possible ratio. Where a specific stereoisomer (e.g. a specific enantiomer) is intended, or a mixture having a specific ratio of stereoisomers is intended, such will be specifically recited. The recognition of stereogenic centers is well-within the skill of a trained organic chemist, and recitation of particular compounds known to have multiple stereogenic centers herein is not intended to exclude by silence those that are not specified discussed herein.

Different polymorphs of the compounds may also be used. Polymorphs are, by definition, crystals of the same molecule having different physical properties as a result of the order of the molecules in the crystal lattice. The polymorphic behavior of drugs can be of crucial importance in pharmacy and pharmacology. The differences in physical properties exhibited by polymorphs affect pharmaceutical parameters such as storage stability, compressibility and density (important in formulation and product manufacturing), and dissolution rates (an important factor in determining bio-availability). Differences in stability can result from changes in chemical reactivity (e.g. differential oxidation, such that a dosage form discolors more rapidly when comprised of one polymorph than when comprised of another polymorph) or mechanical changes (e.g. tablets crumble on storage as a kinetically favored polymorph converts to thermodynamically more stable polymorph) or both (e.g. tablets of one polymorph are more susceptible to breakdown at high humidity). Where a particular polymorphic form of a substance is intended, it will be stated specifically and unambiguously; otherwise, the recitation of a substance is to be interpreted to cover all forms of the substance, including all known polymorphic forms.

Unless otherwise specified or limited herein, recitation of a substance A (wherein A is a variable, such as a mirtazapine, milnacipran, duloxetine, setiptiline, venlafaxine, desvenlafaxine, bicifadine, brofaromine, or a mixture thereof, etc.) is intended to embrace all pharmaceutically acceptable salts, stereoisomers, enantiomers and polymorphs of A that can be made. That is recitation of A is intended to encompass all pharmaceutically acceptable salts of A, all stereoisomers of A, all pharmaceutically acceptable salts of stereoisomers of A, all enantiomers of A, all pharmaceutically acceptable salts of stereoisomers of A, all polymorphs of A, all polymorphs of pharmaceutically acceptable salts of A, all polymorphs of enantiomers of A, all polymorphs of pharmaceutically acceptable salts of stereoisomers of A and all mixtures of the foregoing, which can be made.

A prodrug is a covalently bonded substance which releases the active parent drug in vivo. Prodrugs are prepared by modifying functional groups present in the compound in such a way that the modifications are cleaved, either in routine manipulation or in vivo, to yield the parent compound. Prodrugs include compounds wherein the hydroxy or amino group is bonded to any group that, when the prodrug is administered to a patient, cleaves to form a free hydroxyl or free amino, respectively. Examples of prodrugs include, but are not limited to, acetate, formate and benzoate derivatives of alcohol and amine functional groups.

A metabolite of the above-mentioned compounds results from biochemical processes by which living cells interact with the active parent drug or other formulas or compounds in vivo. Metabolites include products or intermediates from any metabolic pathway.

### Formulations

The compounds, or pharmaceutically acceptable salts thereof, or polymorphic variations thereof, can be formulated as pharmaceutical compositions. Such compositions can be administered orally, buccally, intravenously, parenterally, by inhalation spray, rectally, intradermally, transdermally, pulmonary, nasally or topically in dosage unit formulations containing conventional nontoxic pharmaceutically acceptable carriers, adjuvants, and vehicles as desired. Topical administration may also involve the use of transdermal administration, such as by transdermal patches or iontophoresis devices. The term parenteral as used herein includes subcutaneous, intravenous, intramuscular, or intrasternal injection, or infusion techniques. In the preferred embodiment the composition is administered orally.

Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

The active compounds (or pharmaceutically acceptable salts thereof) may be administered without any additional additives or in the form of a pharmaceutical composition wherein the active compound(s) is in admixture or mixture with one or more pharmaceutically acceptable carriers, excipients or diluents. Pharmaceutical compositions may be formulated in conventional manner using one or more physiologically acceptable carriers comprising excipients and auxiliaries, which facilitate processing of the active compounds into preparations that can be used pharmaceutically. Proper formulation is dependent upon the route of administration chosen. Pharmaceutical compositions may also be coated with one or more suitable coating materials.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name Eudragit® (Roth Pharma, Westerstadt, Germany), zein, shellac, and polysaccharides.

Additionally, the coating material may contain conventional carriers, such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants.

Diluents (that is substances that dilute the active pharmaceutical agent), also referred to as “fillers,” are often used to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone. Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions, which include, by way of example only, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

If desired, the tablets, beads, granules, or particles may also contain minor amount of nontoxic auxiliary substances such as wetting or emulsifying agents, dyes, pH buffering agents, or preservatives.

The compounds may be complexed with other agents as part of their being pharmaceutically formulated. The pharmaceutical compositions may take the form of, for example, tablets or capsules prepared by conventional means with pharmaceutically acceptable excipients such as binding agents (e.g., acacia, methylcellulose, sodium carboxymethylcellulose, polyvinylpyrrolidone (Povidone), hydroxypropyl methylcellulose, sucrose, starch, and ethylcellulose); fillers (e.g., corn starch, gelatin, lactose, acacia, sucrose, microcrystalline cellulose, kaolin, mannitol, dicalcium phosphate, calcium carbonate, sodium chloride, or alginic acid); lubricants (e.g. magnesium stearates, stearic acid, silicone fluid, talc, waxes, oils, and colloidal silica); and disintegrators (e.g. micro-crystalline cellulose, corn starch, sodium starch glycolate and alginic acid). If water-soluble, such formulated complex then may be formulated in an appropriate buffer, for example, phosphate buffered saline or other physiologically compatible solutions. Alternatively, if the resulting complex has poor solubility in aqueous solvents, then it may be formulated with a non-ionic surfactant such as TWEEN™, or polyethylene glycol. Thus, the compounds and their physiologically acceptable solvates may be formulated for administration.

Liquid formulations for oral administration prepared in water or other aqueous vehicles may contain various suspending agents such as methylcellulose, alginates, tragacanth, pectin, kelgin, carrageenan, acacia, polyvinylpyrrolidone, and polyvinyl alcohol. The liquid formulations may also include solutions, emulsions, syrups and elixirs containing, together with the active compound(s), wetting agents, sweeteners, and coloring and flavoring agents. Various liquid and powder formulations can be prepared by conventional methods for inhalation by the patient.

Delayed release and extended release compositions can be prepared. The delayed release/extended release pharmaceutical compositions can be obtained by complexing drug with a pharmaceutically acceptable ion-exchange resin and coating such complexes. Such formulations are coated with a substance that will act as a barrier to control the diffusion of the drug from its core complex into the gastrointestinal fluids. Optionally, the formulation may be coated with a film of a polymer that is insoluble in the acid environment of the stomach, and soluble in the basic environment of lower GI tract in order to obtain a final dosage form that releases less than 10% of the drug dose within the stomach.

In addition, combinations of immediate release compositions and delayed release/extended release compositions may be formulated together.

5HT₂/5HT₃ antagonist/alpha-2 antagonists increase intrasynaptic serotonin and norepinephrine levels. SNRI, NSRI and RIMA compounds increase intrasynaptic serotonin and norepinephrine by different mechanisms of action. The 5HT₂/5HT₃ antagonist/alpha-2 antagonists (such as mirtazapine and setiptiline) increase intrasynaptic NA by blocking alpha-2 sites, while SNRI and NSRI compounds block reuptake of 5-HT at serotonin transporters and block reuptake of norepinephrine at norepinephrine transporters. Thus a combination of 5HT₂/5HT₃ antagonist/alpha-2 antagonist and SNRI, NSRI or RIMA is expected to have a synergistic effect, both by increasing available 5-HT and by increasing available norepinephrine (NA). Accordingly, in some embodiments the invention provides synergistic compositions, which permit effective treatment of patients with lower doses of one, the other or both of 5HT₂/5HT₃ antagonist/alpha-2 antagonist and a second compound selected from SNRI, NSRI and RIMA compounds would normally be indicated for treatment of a disorder. In some embodiments, the invention provides effective treatment of at least one disorder with a dose of both 5HT₂/5HT₃ antagonist/alpha-2 antagonist and SNRI, NSRI or RIMA, wherein the amount of either or both of the 5HT₂/5HT₃ antagonist/alpha-2 antagonist and the SNRI, NSRI or RIMA, is lower than would normally be indicated for either the antagonist or the reuptake inhibitor alone. In some embodiments, the dose of each of the 5HT₂/5HT₃ antagonist/alpha-2 antagonist and the SNRI, NSRI or RIMA is half or less than half of the dose required for either agent alone. In some embodiments, the dose of each of the 5HT₂/5HT₃ antagonist/alpha-2 antagonist and the SNRI, NSRI or RIMA compound is from about 5 to about 45% of the dose required for either agent alone. Thus, in some embodiments, a dosage form provided herein provides about 0.5 to 7.5 mg of mirtazapine and about 5 to 200 mg of duloxetine per dose; and a method according to the invention would comprise administering just one such dosage form to a patient per 24 hour period. In some embodiments, a dosage form provided herein provides about 0.5 to about 5 mg mirtazapine and about 10 mg to about 100 mg of duloxetine per dose; and a method according to the invention would comprise administering just one dosage form to a patient per 24 hour period.

### Immediate 5HT₂/5HT₃ Antagonist/Alpha-2 Antagonist Release and Delayed Release of a SNRI, a NSRI or a RIMA

In some embodiments, formulations combine a SNRI, a NSRI or a RIMA with a 5HT₂/5HT₃ antagonist/alpha-2 antagonist, such as mirtazapine or setiptiline, in a formulation which allows for immediate release of the 5HT₂/5HT₃ antagonist/alpha-2 antagonist and delayed release of the SNRI, NSRI or RIMA. In some embodiments, the SNRI, NSRI or RIMA is not released until at least 6 hours after the 5HT₂/5HT₃ antagonist/alpha-2 antagonist is released. 5HT₂/5HT₃ antagonist/alpha-2 antagonists, such as mirtazapine, may be administered once/day before bed because of the somnolence they produce. Delayed release of the SNRI, NSRI or RIMA ensures that adequate concentrations are available in the circulation following sleep to counteract the excessive daytime sleepiness and/or increased appetite/weight gain associated with the 5HT2/5HT3 antagonist/alpha-2 antagonist.

In such dosages, the 5HT₂/5HT₃ antagonist/alpha-2 antagonist is generally included in an immediate release component (optionally contained within an enteric coating, so that immediate release is affected within the small intestine) and the SNRI, NSRI or RIMA is included in a delayed release component. The two components may be two phases having different release profiles. For example, the two components may be two types of beads or particles contained within an immediate release or enterically coated capsule. The beads may also be pressed together and optionally coated with an immediate release or enteric coating to form a tablet or caplet. The first component, containing 5HT₂/5HT₃ antagonist/alpha-2 antagonist, may be uncoated or coated with a layer of quickly-dissolvable coating material, while the second component, containing the SNRI, NSRI or RIMA, may be coated with a coating that provides from about 1 to about 8, especially about 2 to about 6, hours of delay before the SNRI, NSRI or RIMA is released into the surrounding environment (e.g. the intestines). The second component may also be coated with an enteric coating, thereby ensuring release in the intestines rather than the stomach, and thereby enhancing the delayed action of the second component.

In some embodiments, the unit dose is in the form of a caplet a tablet wherein the second component is in a delayed release layer or core, which may be coated with a delayed release coating and optionally an enteric coating. The first component may then be coated over or layered adjacent to the delayed release core or layer (respectively) in an immediate release layer. This immediate release component layer may be coated with an immediate release coating, an enteric coating (if e.g. the 5HT₂/5HT₃ antagonist/alpha-2 antagonist is acid labile), or both.

In some embodiments, the second component (comprising SNRI, NSRI or RIMA) is a delayed release layer or core comprising a controlled release matrix or osmotic pump. The delayed release layer or core may be coated with a delayed release coating and optionally with an enteric coating. The first therapeutic agent (comprising 5HT₂/5HT₃ antagonist/alpha-2 antagonist) may then be coated over or layered adjacent to the delayed release core or layer in an immediate release component, which may itself be coated with an immediate release coating, an enteric coating (e.g. if the 5HT₂/5HT₃ antagonist/alpha-2 antagonist is acid labile) or both. After the first component quickly dissolves (either in the stomach or in the intestine), the second component remains intact until the delayed release coating dissolves, at which point the controlled release component begins releasing the second therapeutic agent (comprising SNRI, NSRI or RIMA) into the intestines. One skilled in the art will recognize that a controlled release of the second therapeutic agent will provide an increased delay in the attainment of side-effect reducing levels of the second therapeutic agent in the patient's body. Thus, the artisan will recognize that a delayed release coating may be combined with a controlled release matrix or osmotic pump to provide a delay in the onset of stimulation (i.e. counteracting the sedative effect of the 5HT₂/5HT₃ antagonist/alpha-2 antagonist), which in at least some embodiments may be more gradual than would have been provided by a delayed release of greater duration alone.

### Delayed 5HT₂/5HT₃ Antagonist/Alpha-2 Antagonist Release and Immediate SNRI, NSRI or RIMA Release

In some therapeutic settings, it may be more convenient to administer the unit dose upon waking than before bed. Thus, in some embodiments, formulations combine a SNRI, NSRI or RIMA with a 5HT₂/5HT₃ antagonist/alpha-2 antagonist, such as mirtazapine or setiptiline, in a formulation which allows for delayed release of the 5HT₂/5HT₃ antagonist/alpha-2 antagonist and immediate release of the SNRI, NSRI or RIMA. In some embodiments, the 5HT₂/5HT₃ antagonist/alpha-2 antagonist is not released until at least 10 hours after the SNRI, NSRI or RIMA is released. 5HT₂/5HT₃ antagonist/alpha-2 antagonists, such as mirtazapine, are typically administered once/day at night because of the somnolence they produce. Daytime dosing of 5HT₂/5HT₃ antagonist/alpha-2 antagonist would desirably include delaying release of the 5HT₂/5HT₃ antagonist/alpha-2 antagonist component in order to minimize the sedating effects of the 5HT₂/5HT₃ antagonist/alpha-2 antagonist during the day. At the same time immediate release of the SNRI, NSRI or RIMA would counteract the sedative effect of the 5HT₂/5HT₃ antagonist/alpha-2 antagonist during the day and allow this effect to wear off by the time the patient is ready to retire for the night. (It is to be understood that not all patients will follow diurnal sleep patters, so the terms “night” and “day” refer herein to periods including protracted sleep (i.e. at least about 4 hours of sleep) and periods including general wakefulness, respectively.

In such dosages, the SNRI, NSRI or RIMA is generally included in an immediate release component and the 5HT₂/5HT₃ antagonist/alpha-2 antagonist is included in a delayed release component. The two components may be two phases having different release profiles. For example, the two components may be two types of beads or particles contained within an immediate release or enterically coated capsule. The beads may also be pressed together and optionally coated with an immediate release or enteric coating to form a tablet or caplet. The first component, containing SNRI, NSRI or RIMA, may be uncoated or coated with a layer of quickly dissolvable coating material, while the second component, containing the 5HT₂/5HT₃ antagonist/alpha-2 antagonist, may be coated with a coating that provides from about 8 to about 18, especially about 10 to about 12, hours of delay before the 5HT₂/5HT₃ antagonist/alpha-2 antagonist is released into the surrounding environment (e.g. the intestines). The second component may also be coated with an enteric coating, thereby ensuring release in the intestines rather than the stomach, and thereby enhancing the delayed action of the second component.

In some embodiments, the unit dose is in the form of a caplet a tablet wherein the second component is in a delayed release layer or core, which may be coated with a delayed release coating and optionally an enteric coating. The first component may then be coated over or layered adjacent to the delayed release core or layer (respectively) in an immediate release layer. This immediate release component layer may be coated with an immediate release coating, an enteric coating (if e.g. the SNRI, NSRI or RIMA is acid labile), or both.

In some embodiments, the second component (comprising 5HT₂/5HT₃ antagonist/alpha-2 antagonist) is a delayed release layer or core comprising a controlled release matrix or osmotic pump. The delayed release layer or core may be coated with a delayed release coating and optionally with an enteric coating. The first therapeutic agent (comprising SNRI, NSRI or RIMA) may then be coated over or layered adjacent to the delayed release core or layer in an immediate release component, which may itself be coated with an immediate release coating, an enteric coating (e.g. if the SNRI, NSRI or RIMA is acid labile) or both. After the first component quickly dissolves (either in the stomach or in the intestine), the second component remains intact until the delayed release coating dissolves, at which point the controlled release component begins releasing the second therapeutic agent into the intestines. One skilled in the art will recognize that a controlled release of the second therapeutic agent will provide an increased delay in the attainment of a side effect inducing dose of the 5HT₂/5HT₃ antagonist/alpha-2 antagonist in the patient's body. Thus, the artisan will recognize that a delayed release coating may be combined with a controlled release matrix or osmotic pump to provide a delay in the onset of the sedative effect of the 5HT₂/5HT₃ antagonist/alpha-2 antagonist, which in at least some embodiments may be more gradual than would have been provided by a delayed release of greater duration alone.

Delaying the onset of 5HT₂/5HT₃ antagonist/alpha-2 antagonist side effects may also help to reduce weight gain, since the therapeutic dose may be released generally while the patient is sleeping and unable to respond to an increased appetite level by eating.

One skilled in the art will recognize that the 5HT₂/5HT₃ antagonist/alpha-2 antagonist and SNRI, NSRI or RIMA may be combined in single dosage forms having a variety of configurations. For example, the 5HT₂/5HT₃ antagonist/alpha-2 antagonist may be split between an immediate release component and a delayed or delayed/controlled release component. These two 5HT₂/5HT₃ antagonist/alpha-2 antagonist components may be combined with a delayed or delayed/controlled release component comprising a side effect reducing amount of a SNRI, NSRI or RIMA. Such a dosage may be administered immediately before bed or within 0-4 hours before bed, especially about 0-2 hours before bed, and preferably about 0-1 hours before bed. The immediate release of 5HT₂/5HT₃ antagonist/alpha-2 antagonist provides quick attainment of therapeutically effective levels of the antagonist in the body, whereas the delayed or delayed/controlled release of 5HT₂/5HT₃ antagonist/alpha-2 antagonist and delayed or delayed/controlled release of SNRI, NSRI or RIMA counteracts one or more side effects associated with the 5HT₂/5HT₃ antagonist/alpha-2 antagonist, such as sedation and weight gain, while also providing therapeutic 5HT₂/5HT₃ antagonist/alpha-2 antagonist effect throughout the 24 hour period.

The 5HT₂/5HT₃ antagonist/alpha-2 antagonist and the SNRI, NSRI or RIMA may also be combined in a form that provides controlled release of the 5HT₂/5HT₃ antagonist/alpha-2 antagonist (either from a controlled release matrix or controlled release osmotic pump) over a period of time (e.g. from about 10 to about 24 hours) and immediate release of the SNRI, NSRI or RIMA. Such a unit dose would likely be administered from 0-4 hours after waking, e.g. before, after or with a morning meal.

The 5HT₂/5HT₃ antagonist/alpha-2 antagonist and the SNRI, NSRI or RIMA may also be combined in a form that provides controlled release of the 5HT₂/5HT₃ antagonist/alpha-2 antagonist (either from a controlled release matrix or controlled release osmotic pump) over a period of time (e.g. from about 10 to about 24 hours) and delayed or delayed/controlled release of the SNRI, NSRI or RIMA. Such a unit dose would likely be administered from 0-4 hours before bed.

### Disorders to be Treated by the 5HT₂/5HT₃ Antagonist/Alpha-2 Antagonists in Combination with a SNRI, NSRI or RIMA

### Chronic Low Back Pain

Chronic low back pain (CLBP) is a common musculoskeletal disorder that is characterized by pain in the lower back lasting at least 3 months. While a small subset of these patients have existing structural abnormalities or tissue injury, in 90% of CLBP patients the disorder has an unknown etiology. CLBP affects at least 10-15% of the adult population and gives rise to approximately $50 billion in health care costs, disability claims, and lost productivity. Existing drug therapies for CLBP typically provide only marginal or short term benefit and have dose-limiting tolerability issues.

Chronic low back pain is defined as pain, muscle tension, or stiffness localized to the lower back persisting for longer than 3 months. About 10% of the cases originate from injuries or degeneration of spinal structures including muscle-ligament injuries, disk herniation, and spinal stenosis. Approximately 90% of cases, however, have no identifiable cause or anatomical abnormalities that clearly explain their symptoms and are designated nonspecific or idiopathic. Manek, N. J. and A. J. MacGregor, Epidemiology of back disorders: prevalence, risk factors, and prognosis. Curr. Opin. Rheumatol., 2005. 17(2): p. 134-40. Nonspecific terms such as strain, sprain, or degenerative processes are commonly used. Diagnostic evaluation is often frustrating for both physicians and patients because a precise anatomical explanation is elusive. Some experts [see, Praemer, A., Furnes, S., Rice, D. P., Musculoskeletal conditions in the United States. 1992: p. 1-99.] suggest it is generally more useful for the physician to address 3 questions: Is a systemic disease causing the pain? Is there social or psychological distress that may amplify or prolong the pain? Is there neurological compromise that may require surgical evaluation? These questions can be addressed through medical history and physical examination., which is within the skill of the typical practitioner.

Point prevalence estimates showed 6.4% of the population suffers from chronic low back pain. Han et al., 2000. Chronic low back pain is a leading reason for physician visits and work disability and costs the U.S. over $50 billion annually in health care costs, disability claims, and lost productivity. Frymoyer, J. W. and W. L. Cats-Baril, An overview of the incidences and costs of low back pain. Orthop. Clin. North Am., 1991. 22(2): p. 263-71.

Risk factors for chronic low back pain include those within the individual, occupational, and psychosocial domains. See Manek, 2005. Individual risk factors include smoking, obesity, and age. Although the prevalence of chronic low back pain increases with age, the dose-response relation between age and low back pain is not linear, suggesting multiple factors are involved. Women, but not men, who are overweight or with large hip-to-waist ratios have an increased likelihood of developing chronic low back pain. Suzuki et al., 2004.

Sleep disturbances and complaints of poor sleep quality are very common among patients with pain-related conditions. Additionally, sleep improvement is often used as an indicator of pain relief. In chronic low back pain, subjective measures indicate the presence and stability of sleep disturbance; although, objective assessments using polysomnography revealed only subtle differences in sleep quality. Harman, K., et al., Sleep in depressed and nondepressed participants with chronic low back pain: electroencephalographic and behavior findings. Sleep, 2002. 25(7): p. 775-83. Hence, agents that improve sleep could have a beneficial effect in chronic low back pain patients. On the other hand, sedative agents can have deleterious effects on patients during waking hours, interfering with normal activities as well as the operation of heavy equipment, including automobiles. Thus therapeutics that promote sleep but induce daytime sleepiness are considered unsuitable for long term care in many circumstances.

As discussed previously herein, neural pathways originating from the brainstem suppress sensory transmission and consequently produce analgesia. Suzuki et al., 2004. These descending inhibitory pathways typically utilize 5-HT and NA as neurotransmitters, and this may partially explain why drugs that enhance extracellular levels of 5-HT and NA, such as the tricyclic antidepressants have been found clinically to exhibit analgesic properties in chronic pain conditions.

Central sensitization is a CNS condition that typically occurs following peripheral nerve injury, and consequently neurons in the spinal cord become hyperexcitable and much more responsive to neuronal inputs from the periphery. Suzuki et al., 2004. These inputs are usually too weak to cause excitation under normal circumstances, but in sensitized states, non-noxious stimuli can lead to widespread pain extending beyond the site of damage/stimulation. In chronic low back pain, it has been hypothesized that a process somewhat similar to central sensitization may be responsible for the heightened and long-term pain that occurs in the absence of sustained tissue injury. Arendt-Nielsen, L. and T. Graven-Nielsen, Central sensitization in fibromyalgia and other musculoskeletal disorders. Curr Pain Headache Rep, 2003. 7(5): p. 355-61.

Ion channels also play an important role in mediating chronic pain states. Activation or increased expression of Na⁺ and Ca⁺⁺ channels enhances membrane excitability directly leading to increased neuronal signaling. Nerve injury, which can produce chronic pain, enhances the expression of Na⁺ channels. Priestly et al., 2004. Blockade of Na⁺ channels with lidocaine reduces pain associated with nerve injury both in animal models and in humans. Similarly, Ca⁺⁺ channel subunit expression is also increased following nerve injury and the Ca⁺⁺ channel blocker ziconotide reduces pain in animals and humans. McGivem, J. G. and S. I. McDonough, Voltage-gated calcium channels as targets for the treatment of chronic pain. Curr. Drug Targets CNS Neurol. Disord., 2004. 3(6): p. 457-78.

Specifically, some embodiments provided herein provide that mirtazapine, which has the ability to elevate serotonin (“5-HT”) and norepinephrine (“NA”), is combined with milnacipran, which also elevates 5-HT and NA by inhibiting their reuptake, resulting in enhanced pain reduction. Additionally, as milnacipran counteracts the sedative and orexigenic effects of SNRI, NSRI or RIMAs, the combination of 5HT₂/5HT₃ antagonist/alpha-2 antagonist and milnacipran is expected to result in an improved side effect profile as compared to a 5HT₂/5HT₃ antagonist/alpha-2 antagonist by itself. In some embodiments this treatment regime is beneficial, especially where treatment of chronic low back pain is particularly intractable. Thus, it is a further object to provide analgesia while reducing or avoiding the side-effects associated with use of mirtazapine alone, such as sedation, excessive daytime sleepiness and weight gain.

Mirtazapine's ability to elevate 5-HT and NA suggests its utility in treating chronic pain because drugs with similar effects (tricyclic antidepressants, NSRIs) produce beneficial responses in alleviating chronic pain. Although there are no published trials in chronic low back pain, an open study suggested that mirtazapine has some activity in fibromyalgia pain [Samborski, W., M. Lezanska-Szpera, and J. K. Rybakowski, Open trial of mirtazapine in patients with fibromyalgia. Pharmacopsychiatry, 2004. 37(4): p. 168-70] and a controlled trial suggested efficacy in chronic tension type headache. Bendtsen, L. and R. Jensen, Mirtazapine is effective in the prophylactic treatment of chronic tension-type headache. Neurology, 2004. 62(10): p. 1706-11. An open trial in cancer pain, however, found that, although significant improvements were obtained in depression levels, pain intensity was not statistically improved, although a trend for improvement was apparent. Theobald, D. E., et al., An open-label, crossover trial of mirtazapine (15 and 30 mg) in cancer patients with pain and other distressing symptoms. J Pain Symptom Manage, 2002. 23(5): p. 442-7. In addition to mirtazapine's ability to elevate both 5-HT and NA, its 5HT₃ blocking activity could also useful for reducing pain, based on clinical studies with the 5HT₃ antagonist tropisetron in low back pain.

As mentioned above, poor sleep quality is a common complaint of patients with chronic low back pain. Mirtazapine has sleep-promoting properties and is often prescribed to depressed patients with insomnia. Clinical studies have found that mirtazapine improves objective and subjective sleep measures, in both depressed patients and in normal subjects, including sleep onset, total sleep time, and sleep efficiency. Aslan, S., E. Isik, and B. Cosar, The effects of mirtazapine on sleep: a placebo controlled, double-blind study in young healthy volunteers. Sleep, 2002. 25(6): p. 677-9. Winokur, A., et al., Acute effects of mirtazapine on sleep continuity and sleep architecture in depressed patients: a pilot study. Biol. Psychiatry, 2000. 106(1): p. 75-8. The analgesic and somnolence-promoting effects of mirtazapine may provide additive benefits in chronic low back pain.

Mirtazapine has been associated with increases in appetite and body weight. In controlled studies, appetite increase was reported in 17% of patients treated with mirtazapine, compared to 2% for placebo, and 6% for amitriptyline. In these same trials, weight gain of greater than or equal to 7% of body weight was reported in 7.5% of patients treated with mirtazapine, compared to 0% for placebo and 5.9% for amitriptyline. Results from a long-term trial with mirtazapine in depressed patients suggests that the greatest weight gain occurs during the initial 12 weeks of treatment with only slight weight gain occurring during the 40 week extension phase. Krishnan, K. R. 2004, personal communication. While mirtazapine may increase appetite and body weight when administered alone, milnacipran or duloxetine in combination with mirtazapine may counteract this potential adverse effect.

In some embodiments, then 5HT₂/5HT₃ antagonist/alpha-2 antagonist and SNRI, NSRI or RIMA in combination provide superior analgesia in patients with chronic low back pain. For example combination of mirtazapine or a pharmaceutically acceptable salt thereof with milnacipran or duloxetine is contemplated. Another example would be setiptiline and milnacipran or duloxetine. Another example would be mirtazapine and atomoxetine as the free base or a pharmaceutically acceptable salt thereof. Another example would be setiptiline with atomoxetine or a pharmaceutically acceptable salt thereof. Moreover, in some embodiments mirtazapine and milnacipran or duloxetine in combination have limited side effects compared to other drugs currently in use, or at least reduced side effects when compared to either drug taken separately.

### Sleep-Related Breathing Disorders

Over the past several years much effort has been devoted to the study of a discrete group of breathing disorders that occur primarily during sleep with consequences that may persist throughout the waking hours in the form of sleepiness, thereby manifesting itself into substantial economic loss (e.g., thousands of lost man-hours) or employment safety factors (e.g., employee non-attentiveness during operation of heavy-machinery). Sleep-related breathing disorders are characterized by repetitive reduction in breathing (hypopnea), snoring, periodic cessation of breathing (apnea), or a continuous or sustained reduction in ventilation.

In general sleep apnea is defined as an intermittent cessation of airflow at the nose and mouth during sleep. Sleep apnea has been linked to serious medical conditions such as heart disease, hypertension, stroke, obesity, and decreased pulmonary function. In severe cases sleep apnea may even cause death. By convention, apneas of at least 10 seconds in duration have been considered important, but in most individuals the apneas are 20-30 seconds in duration and may be as long as 2-3 minutes. While there is some uncertainty as to the minimum number of apneas that should be considered clinically important, by the time most individuals come to attention of the medical community they have at least 10 to 15 events per hour of sleep.

Sleep apneas have been classified into three types: central, obstructive, and mixed. In central sleep apnea the neural drive to all respiratory muscles is transiently abolished. In obstructive sleep apneas (OSAS), airflow ceases despite continuing respiratory drive because of occlusion of the oropharyngeal airway. Mixed apneas, which consist of a central apnea followed by an obstructive component, are a variant of obstructive sleep apnea. The most common type of apnea is obstructive sleep apnea.

Hypopneas are episodes of shallow breathing during which airflow is decreased by at least 50%. Like apnea, hypopnea is subdivided as being obstructive, central, or mixed. Obstructive hypopneas are episodes of partial upper airway obstruction. In central hypopnea, breathing effort and airflow are both decreased. Mixed hypopneas have both central and obstructive components. Individuals with OSA syndrome have pathologic degrees of obstructive apnea, obstructive hypopnea, or both.

The term Upper Airway Resistance Syndrome (UARS) is used to describe chronic daytime sleepiness in the absence of actual apneas or hypopneas, but often associated with snoring, and with brief, frequent arousals with an only slightly abnormal breathing pattern. Patients with the clinical features of apnea, hypopnea and nocturnal oxygen desaturation during polysomnography (PSG).

Patients with UARS lack the typical findings of apnea on PSG, and therefore, are often not diagnosed. The arousals and sleep fragmentation are related to an increased effort to breathe which can be diagnosed by measurement of pressure changes in the esophagus.

The term “snoring” generally refers to a rough or hoarse sound that arises from a person's mouth during sleep. Snoring is believed to be generally caused by the narrowing of the pharyngeal airway such that turbulent airflow during relaxed breathing vibrates the soft parts of the pharyngeal passage, such as the soft palate, the posterior faucial pillars of the tonsils and the uvula. A restricted pharyngeal passageway can occur anatomically. For example, in children, this often is caused by obstruction due to enlarged tonsils or adenoids. In adults, it is not unusual for the narrowing to be caused by obesity. Further anatomical narrowing can be simple a matter of heredity, with some persons being predisposed towards a smaller pharyngeal cross-section. A reduced pharyngeal passageway may also be caused by a lack of muscle tone.

Snoring can indicate a more serious condition and, due to exhaustion resulting from lack of sleep, can cause other problems. For example, an association between snoring and coronary artery disease and hypertension has been found, and cardiac arrhythmia has been reported during sleep apnea attacks. As stated above, people with sleep apnea often snore, however, sleep apnea can also be present without snoring. Not only is the risk of cessation of breathing dangerous, lack of oxygen due to an obstructed pharyngeal passageway deprives the body of sufficient oxygen so that oxygen desaturation arises. Lack of oxygen may cause the brain to rouse the sleeper just enough to take a breath without fully awaking. This may occur hundreds of times a night, with the result that the snorer fails to get sufficient sleep. Moreover, being aroused from deep REM sleep on a repetitive basis may increase heart rate and blood pressure. Thus, snoring may increase the risk of heart attack and stroke (Leineweber et al. Sleep 27(7): 1344-1349 (2004)).

### Depression

Depression refers to an abnormal mood or a collection of symptoms that constitute a psychiatric disorder. Symptoms of depression include disturbances in mood and affect (depressed mood, diminished interest and pleasure in activities), bodily function (weight and appetite changes, psychomotor disturbances, sleep disturbances, fatigue and loss of energy), and cognitive processes (feelings of worthlessness and guilt, concentration difficulties, indecisiveness, thoughts of death or suicide and possibly delusions/hallucinations). These symptoms vary in intensity, duration and frequency and permit classification of depression into different classes. Other symptoms of major depressive episodes include crying spells, self-pity, hopelessness, irritability, brooding, diminished self-esteem, decreased libido, nihilism, social withdrawal, memory impairment, feelings of inadequacy and pessimism. These symptoms are summarized in the American Psychiatric Association's Diagnostic and Statistical Manual of Mental Disorders, 4th Edition, Text Revision (DSM-IV-TR; 1994).

Atypical depression is one type of depressive disorder included in DSM-IV-TR at page 420 about which there has been substantial clinical and research interest. Although at the present time it is not clear how common this diagnosis is in chronic pain patients, there are certainly pain patients expressing the characteristics of atypical depression.

There are at least two broad types of atypical depression that differ from classically defined depression (Davidson et al. Arch. Gen. Psychiatry, 39, 527-34 (1982); Paykel et al. Psychol. Med., 13, 131-9 (1983); Paykel et al, Arch. Gen. Psychiatry, 39:1041-9 (1982)). One is composed of those depressions accompanied by severe anxiety, and also by phobic symptoms, tension, and pain. The other type of atypical depression is characterized by reversed vegetative symptoms, e.g., increased (rather than decreased) appetite, weight, and sleep.

### Schizophrenia

Schizophrenia is a devastating brain disorder that affects approximately 2.2 million American adults, or 1.1 percent of the population age 18 and older. Schizophrenia interferes with a person's ability to think clearly, to distinguish reality from fantasy, to manage emotions, make decisions, and relate to others. The first signs of schizophrenia typically emerge in the teenage years or early twenties. Most people with schizophrenia suffer chronically or episodically throughout their lives, and are often stigmatized by lack of public understanding about the disease.

The symptoms of schizophrenia are generally divided into three categories, including positive, disorganized and negative symptoms. Positive Symptoms, or “psychotic” symptoms, include delusions and hallucinations because the patient has lost touch with reality in certain important ways. Disorganized Symptoms include confused thinking and speech, and behavior that does not make sense. Negative Symptoms include emotional flatness or lack of expression, an inability to start and follow through with activities, speech that is brief and lacks content, and a lack of pleasure or interest in life.

Schizophrenia is also associated with changes in cognition. These changes affect the ability to remember and to plan for achieving goals. Attention and motivation are also diminished. The cognitive problems of schizophrenia may be important factors in long term outcome.

Schizophrenia also affects mood. Many individuals affected with schizophrenia become depressed, and some individuals also have apparent mood swings and even bipolar-like states. When mood instability is a major feature of the illness, it is called, schizoaffective disorder, meaning that elements of schizophrenia and mood disorders are prominently displayed by the same individual. It is not clear whether schizoaffective disorder is a distinct condition or simply a subtype of schizophrenia.

### Anxiety Disorders

Generalized Anxiety Disorder

Most people experience anxiety at some point in their lives and some nervousness in anticipation of a real situation. However if a person cannot shake unwarranted worries, or if the feelings are jarring to the point of avoiding everyday activities, he or she most likely has an anxiety disorder. Symptoms include chronic, exaggerated worry, tension, and irritability that appear to have no cause or are more intense than the situation warrants. Physical signs, such as restlessness, trouble falling or staying asleep, headaches, trembling, twitching, muscle tension, or sweating, often accompany these psychological symptoms.

Panic Disorder

People with panic disorder experience white-knuckled, heart-pounding terror that strikes suddenly and without warning. Since they cannot predict when a panic attack will seize them, many people live in persistent worry that another one could overcome them at any moment. Symptoms include pounding heart, chest pains, lightheadedness or dizziness, nausea, shortness of breath, shaking or trembling, choking, fear of dying, sweating, feelings of unreality, numbness or tingling, hot flashes or chills, and a feeling of going out of control or going crazy.

Phobias

Phobias are irrational fears that lead people to altogether avoid specific things or situations that trigger intense anxiety. Phobias occur in several forms, for example, agoraphobia is the fear of being in any situation that might trigger a panic attack and from which escape might be difficult. Social Phobia or Social Anxiety Disorder is the fear of social situations and the interaction with other people, which can automatically bring on feelings of self-consciousness, judgment, evaluation, and criticism. It is the fear and anxiety of being judged and evaluated negatively by other people, leading to feelings of inadequacy, embarrassment, humiliation, and depression. Many of the physical symptoms that accompany panic attacks—such as sweating, racing heart, and trembling—also occur with phobias.

Post-Traumatic Stress Disorder

Anyone can develop Post-traumatic Stress Disorder (PTSD) if they have experienced, witnessed, or participated in a traumatic occurrence-especially if the event was life threatening. PTSD can result from terrifying experiences such as rape, kidnapping, natural disasters, war or serious accidents such as airplane crashes. The psychological damage such incidents cause can interfere with a person's ability to hold a job or to develop intimate relationships with others. The symptoms of PTSD can range from constantly reliving the event to a general emotional numbing. Persistent anxiety, exaggerated startle reactions, difficulty concentrating, nightmares, and insomnia are common. People with PTSD typically avoid situations that remind them of the traumatic event, because they provoke intense distress or even panic attacks.

Insomnia

Insomnia is chronic and persistent difficulty in either (1) falling asleep (initial insomnia), (2) remaining asleep through the night (middle insomnia), or (3) waking up too early (terminal insomnia). All types of insomnia can lead to daytime drowsiness, poor concentration, and the inability to feel refreshed and rested in the morning.

There are several types of insomnia. Sleep-onset insomnia occurs when people have difficulty falling asleep because they think and worry and cannot let their minds relax. Sleep maintenance insomnia occurs when people fall asleep normally but wake up several hours later and cannot fall asleep again easily. Sometimes they drift in and out of a restless, unsatisfactory sleep. Early morning awakening, another type of insomnia, may be a sign of depression in people of any age.

Sleep-wake schedule disorder may occur in people whose sleep patterns have been disrupted: They fall asleep at inappropriate times and then cannot sleep when they should. These sleep-wake reversals often result from jet lag (especially when traveling from east to west), working irregular night shifts, frequent changes in work hours, or excessive use of alcohol. Sometimes sleep-wake reversals are a side effect of drugs. Sleep-wake reversals are common among people who are hospitalized because they are often awakened during the night. Damage to the brain's built-in biologic clock (caused by encephalitis, stroke, or Alzheimer's disease, for example) can also disrupt sleep patterns.

### Headaches

Tension-Type headaches

Tension type headaches are the most common, affecting upwards of 75% of all headache sufferers. Tension-type headaches are typically a steady ache rather than a throbbing one and affect both sides of the head. Tension-type headaches may also be chronic, occurring frequently or even every day.

Migraine Headaches

Migraine headaches are less common than tension-type headaches. Nevertheless, migraines afflict 25 to 30 million people in the United States alone. Migraines are felt on one side of the head by about 60% of migraine sufferers, and the pain is typically throbbing in nature. Migraines are often accompanied by nausea and sensitivity to light and sound. A group of telltale neurologic symptoms known as an aura, sometimes occurs before the head pain begins. Typically, an aura involves a disturbance in vision that may consist of brightly colored or blinking lights in a pattern that moves across the field of vision. Usually, migraine attacks are occasional, or sometimes as often as once or twice a week, but not daily.

Cluster Headaches

Cluster headaches are relatively rare, affecting about 1% of the population, and are distinct from migraine and tension-type headaches. Cluster headaches come in groups or clusters lasting weeks or month. The pain is extremely severe, but the attack is brief, lasting no more than an hour or two. The pain centers around one eye, and this eye may be inflamed and watery. There may also be nasal congestion on the affected side of the face. These headaches may strike in the middle of the night, and often occur at about the same time each day during the course of a cluster.

Hot Flashes

Approximately 143% of women will experience hot flashes to some degree. Also known as hot flushes, these symptoms appear because of changing hormone levels around the time of menopause. For some women, hot flashes are nothing more than a mild and fleeting sensation of warmth, but for others hot flashes cause frequent, intense discomfort. Typically, a hot flash starts with increased blood flow to the extremities, increased heart rate and anxiety. A noticeable flush appears on the face and chest, and the sensation of heat may be pronounced. The profuse sweating that often accompanies a hot flash can be a source of stress and social embarrassment, and may interfere with restful sleep.

The precise mechanism responsible for hot flashes is not known for certain, but hormone fluctuations are thought to be a significant factor. Underweight women tend to experience more frequent hot flashes, possibly because fat plays a supportive role in hormone production. In addition, hot flashes affect smokers earlier in life than nonsmokers.

### Functional Somatic Syndromes

Chronic Fatigue Syndrome

Chronic fatigue syndrome (CFS) is a debilitating disorder characterized by profound tiredness or fatigue. Patients with CFS may become exhausted with only light physical exertion, and must often function at a level of activity substantially lower than their capacity before the onset of illness. In addition to the key defining characteristic of fatigue, CFS patients generally report various nonspecific symptoms, including weakness, muscle aches and pains, excessive sleep, malaise, fever, sore throat, tender lymph nodes, impaired memory and/or mental concentration, insomnia, and depression. Like patients with fibromyalgia, patients with CFS suffer from disordered sleep, localized tenderness, and complaints of diffuse pain and fatigue.

There are two widely used criteria for diagnosing CFS. The criteria established by the U.S. Centers for Disease Control and Prevention include medically unexplained fatigue of at least six months duration that is of new onset, not a result of ongoing exertion and not substantially alleviated by rest, and a substantial reduction in previous levels of activity. In addition, the diagnosis involves the determination of the presence of four or more of the following symptoms—subjective memory impairment, tender lymph nodes, muscle pain, joint pain, headache, unrefreshing sleep, and postexertional malaise (>24 hours) (Reid et al., 2000, British Medical Journal 320: 292-296). The diagnostic criteria from Oxford includes severe, disabling fatigue of at least six months duration that affects both physical and mental functioning and the fatigue being present for more than 50% of the time. In addition, the diagnosis involves the determination of the presence of other symptoms, particularly myalgia and sleep and mood disturbance (Reid et al., 2000, British Medical Journal 320: 292-296).

Fibromyalgia Syndrome

Fibromyalgia syndrome (FMS) is the most frequent cause of chronic, widespread pain, estimated to affect 2-4% of the population. FMS is characterized by a generalized heightened perception of sensory stimuli. Patients with FMS display abnormalities in pain perception in the form of both allodynia (pain with innocuous stimulation) and hyperalgesia (increased sensitivity to painful stimuli). The syndrome, as defined by the American College of Rheumatology's criteria, involves the presence of pain for over 3 months duration in all four quadrants of the body, as well as along the spine. In addition, pain is elicited at 11 out of 18 “tender points” upon palpation. Other associated symptoms include fatigue, nonrestorative sleep, and memory difficulties.

Owing to their common symptomology, FMS and CFS are thought to be related. However, they manifest different major symptoms. Whereas pain is the major symptom reported by patients with FMS, fatigue is the major symptom reported by patients with CFS. Given their relatedness, these two indications have been treated with the same medications.

Irritable Bowel Syndrome

Irritable bowel syndrome (IBS) is a gastrointestinal disorder characterized by continuous or recurrent abdominal pain or discomfort that is relieved with defecation and is associated with a change in the consistency or frequency of stool. IBS has elements of an intestinal motility disorder, a visceral sensation disorder, and a central nervous disorder. While the symptoms of IBS have a physiological basis, no physiological mechanism unique to IBS has been identified. Epidemiological surveys have estimated the prevalence of IBS ranges from 10-22% of the population with a higher frequency of occurrence in women. Psychological factors, either stress or overt psychological disease, modulate and exacerbate the physiological mechanisms that operate in IBS (Drossman, D. A. et al., Gastroenterology 1988 95:701-708).

Due to a lack of readily identifiable structural or biochemical abnormalities in this syndrome, the medical community has developed a consensus definition and criteria, known as the Rome criteria, to aid in diagnosis of IBS. According to the Rome criteria, IBS is indicated by abdominal pain or discomfort which is (1) relieved by defection and/or (2) associated with a change in frequency or consistency of stools, plus two or more of the following: altered stool frequency, altered stool form, altered stool passage, passage of mucus, and bloating or feeling of abdominal distention (Dalton, C. and Drossman, D. A., Am. Fam. Physician 1997 55(3):875-880). Thus, a hallmark of IBS is abdominal pain that is relieved by defecation, and which is associated with a change in the consistency or frequency of stools. IBS may be diarrhea-predominant, constipation-predominant, or an alternating combination of both.

Non-gastrointestinal symptoms are common and increase in number as the severity of IBS increases. Chronic fatigue, headache, urological symptoms and other multi-system complaints occur including fibromyalgia. In some preferred embodiments, the combination of a 5HT₂/5HT₃ antagonist/alpha-2 antagonist and a SNRI, NSRI or RIMA is useful for the treatment of diarrhea predominant IBS.

Lower Back Pain (Other than Chronic Lower Back Pain)

Aside from chronic lower back pain, which is a functional somatic disorder, other common causes of lower back pain include lumbar strain, nerve irritation, lumbar radiculopathy, bony encroachment, and conditions of the bone and joints.

Lumbar Strain—A lumbar strain is a stretching injury to the ligaments, tendons, and/or muscles of the lower back. The stretching incident results in microscopic tears of varying degrees in these tissues. Lumbar strain is considered one of the most common causes of low back pain. The injury can occur because of overuse, improper use, or trauma. Soft tissue injury is commonly classified as “acute” if it has been present for days to weeks. If the strain lasts longer than 3 months, it is referred to as “chronic.” Lumbar strain most often occurs in persons in their forties, but can happen at any age. The condition is characterized by localized discomfort in the lower back area with onset after an event that mechanically stressed the lumbar tissues. The severity of the injury ranges from mild to severe, depending on the degree of strain and resulting spasm of the muscles of the lower back.

Nerve Irritation—The nerves of the lumbar spine can be irritated by mechanical impingement or disease any where along their paths—from their roots at the spinal cord to the skin surface. These conditions include lumbar disc disease (radiculopathy), bony encroachment, and inflammation of the nerves caused by a viral infection (shingles).

Lumbar Radiculopathy—Lumbar radiculopathy refers to nerve irritation which is caused by damage to the discs between the vertebrae. Damage to the disc occurs because of degeneration (“wear and tear”) of the outer ring of the disc, traumatic injury, or both. As a result, the central softer portion of the disc can rupture (herniate) through the outer ring of the disc and abut the spinal cord or its nerves as they exit the bony spinal column. This rupture is what causes the commonly recognized “sciatica” pain that shoots down the leg. Sciatica can be preceded by a history of localized low back aching or it can follow a “popping” sensation and be accompanied by numbness and tingling. The pain commonly increases with movements at the waist and can increase with coughing or sneezing. In more severe instances, sciatica can be accompanied by incontinence of the bladder and/or bowels.

Bony Encroachment—Any condition that results in movement or growth of the vertebrae of the lumbar spine can limit the space (encroachment) for the adjacent spinal cord and nerves. Causes of bony encroachment of the spinal nerves include foraminal narrowing (narrowing of the portal through which the spinal nerve passes from the spinal column, out of the spinal canal to the body), spondylolisthesis (slippage of one vertebra relative to another), and spinal stenosis (compression of the nerve roots or spinal cord by bony spurs or other soft tissues in the spinal canal). Spinal nerve compression in these conditions can lead to sciatica pain which radiates down the lower extremities. Spinal stenosis can cause lower extremity pains which worsen with walking and are relieved by resting (mimicking poor circulation).

Bone & Joint Conditions—Bone and joint conditions that lead to low back pain include those existing from birth (congenital), those that result from wear and tear (degenerative) or injury, and those that are from inflammation of the joints (arthritis).

Congenital causes (existing from birth) of low back pain include scoliosis and spina bifida. Scoliosis is a sideways (lateral) curvature of the spine which can be caused when one lower extremity is shorter than the other (functional scoliosis) or because of an abnormal design of the spine (structural scoliosis). Spina bifida is a birth defect in the bony vertebral arch over the spinal canal, often with absence of the spinous process. This birth defect most commonly affects the lowest lumbar vertebra and the top of the sacrum.

As we age, the water and protein content of the body's cartilage changes. This change results in weaker, thinner, and more fragile cartilage. Because both the discs and the joints that stack the vertebrae (facet joints) are partly composed of cartilage, these areas are subject to wear and tear over time (degenerative changes). Degeneration of the disc is called spondylosis. Spondylosis can be noted on x-rays of the spine as a narrowing of the normal “disc space” between the vertebrae. It is the deterioration of the disc tissue that predisposes the disc to herniation and localized lumbar pain (“lumbago”) in older patients. Degenerative arthritis (osteoarthritis) of the facet joints is also a cause of localized lumbar pain that can be detected with plain x-ray testing. These causes of degenerative back pain are usually treated conservatively with intermittent heat, rest, rehabilitative exercises, and medications to relieve pain, muscle spasm, and inflammation.

Fractures (breakage of bone) of the lumbar spine and sacrum bone most commonly affect elderly persons with osteoporosis, especially those who have taken long-term cortisone medication. For these individuals, occasionally even minimal stresses on the spine (such as bending to tie shoes) can lead to bone fracture. In this setting, the vertebra can collapse (vertebral compression fracture). The fracture causes an immediate onset of severe localized pain that can radiate around the waist in a band-like fashion and is made intensely worse with body motions.

The spondyloarthropathies are inflammatory types of arthritis that can affect the lower back and sacroiliac joints. Examples of spondyloarthropathies include Reiter's disease, ankylosing spondylitis, psoriatic arthritis, and the arthritis of inflammatory bowel disease. Each of these diseases can lead to pain and stiffness in the lower back which is typically worse in the morning. These conditions usually begin in the second and third decades of life.

Neuropathic Pain

Neuropathic pain (e.g. from diabetic peripheral neuropathy) may result from a wide spectrum of insults to the peripheral or central nervous system. This may include nutritional deficiencies, systemic diseases, chemotherapy, cerebrovascular accident, surgery or trauma. The hallmark of neuropathic pain is abnormal neural activity in peripheral nerve(s) or the central nervous system. This is often accompanied by disordered sensory processing both in the peripheral or central nervous system. Even in injuries which are primarily peripheral in their location, the central nervous system often becomes involved. The pain frequently has burning, lancinating, or electric shock qualities. Persistent allodynia, pain resulting from a non-painful stimulus such as a light touch, is also a common characteristic of neuropathic pain. The pain may persist for months or years beyond the apparent healing of any damaged tissues.

Side Effects Associated with 5HT₂/5HT₃ Antagonist/Alpha-2 Antagonists

The side effects associated with 5HT₂/5HT₃ antagonist/alpha-2 antagonists include somnolence (sedation, excessive daytime sleepiness, etc.) and orexigenic effects (excessive appetite, weight gain, etc.)

### Excessive Daytime Sleepiness and Weight Gain

Mirtazapine use in the treatment of disorders such as depression, schizophrenia, anxiety disorders, affective disorders, sleep-related breathing disorders, insomnia, migraine headache, chronic tension-type headache, hot flashes, and functional somatic syndromes can cause excessive daytime sleepiness and weight gain in a patient by its sedating effects. The drug is usually given at night, however, because of its long half-life, it can cause sleepiness or fatigue during the day. This often contributes to weight gain by reducing an individual's daily physical activity level.

The symptoms of excessive daytime sleepiness include an overwhelming desire to sleep during what should be waking hours, the need for frequent naps, the inability to concentrate, falling asleep during meetings, class, at work or driving. People find that excessive daytime sleepiness can interfere with their ability to be productive and maintain healthy social relationships. They sometimes feel low self-esteem, frustration, and anger at oneself caused by the disorder and are sometimes misunderstood as being lazy or unintelligent.

### Methods of Use

### Administration Protocol

The 5HT₂/5HT₃ antagonist/alpha-2 antagonist compositions are administered in an effective dosage to alleviate the symptoms of a disorder and the SNRI, NSRI or RIMA is administered in combination with the 5HT₂/5HT₃ antagonist/alpha-2 antagonist in an effective dosage to reduce the side effects associated with the 5HT₂/5HT₃ antagonist/alpha-2 antagonist. The compositions will preferably be administered orally. In one embodiment, the 5HT₂/5HT₃ antagonist/alpha-2 antagonist and SNRI, NSRI or RIMA are administered simultaneously, e.g. in a combination as described herein. In another embodiment, the SNRI, NSRI or RIMA is not administered until at least 6 hours after the 5HT₂/5HT₃ antagonist/alpha-2 antagonist. The compositions can be administered as immediate release, sustained release, intermittent release, and/or delayed release formulations, as described in more detail herein. The composition can be administered in a single dose, an escalating dose, or administered at an elevated dosage which is then decreased to a lower dosage after a particular circulating blood concentration of the compound has been achieved.

An intermittent administration protocol may be used where chronic administration is not desirable. The compound or formulation is administered in time blocks of several days with a defined minimum washout time between blocks. Intermittent administration occurs over a period of several weeks to months to achieve a significant improvement in the symptoms of the disorders.

One of skill in the art would be able to choose administration protocols and determine appropriate dosing regimes to treat symptoms of sleep-related breathing disorders based on bioavailability and half-life of the compound to be administered. For many of the disclosed compounds, appropriate dosage ranges have been established to maximize circulating concentrations of the compound and minimize side-effects.

The 5HT₂/5HT₃ antagonist/alpha-2 antagonist can be administered for a specific duration to improve symptoms of a particular disorder. A suitable endpoint can be where one symptom of the disorder is treated by administration of the compound and the treatment considered effective. In other situations, the treatment can be considered effective when more than one symptom is treated. The SNRI, NSRI or RIMA can be administered in combination with the 5HT₂/5HT₃ antagonist/alpha-2 antagonist for the duration of use of the 5HT₂/5HT₃ antagonist/alpha-2 antagonist or even after treatment has been discontinued. A suitable endpoint can be where one side effect of the 5HT₂/5HT₃ antagonist/alpha-2 antagonist is treated by administration of the SNRI, NSRI or RIMA and the treatment is considered effective. In other situations, the treatment can be considered effective when more than one side effect is treated.

Effective Dosage Ranges

Appropriate dosages can be determined by one of skill in the art based on using routine experimentation and standard techniques utilizing dosages currently approved. Compounds in the disclosed drug classes are known in the art and can be initially administered at similar doses and titrated appropriately to treat symptoms of the disorders and side effects in a given patient. Intra-patient variability is known in the art depending on the severity of symptoms and dosages are commonly adjusted to exact a particular therapeutic effect in a particular patient.

Therapeutically effective amounts for use in humans can also be determined from animal models. For example, a dose for humans can be formulated to achieve a circulating concentration that has been found to be effective in animals. Effective amounts for use in humans can also be determined from human data for the compounds used to treat other disorders, for example, neurological disorders. The amount administered can be the same amount administered to treat other neurological disorders or can be an amount higher or lower than the amount administered to treat other neurological disorders.

The optimal concentration of the drug in each pharmaceutical formulation varies according to the formulation itself. Typically, the pharmaceutical formulation contains the drug at a concentration of about 0.1 to 90% by weight (such as about 1-20% or 1-10%). Appropriate dosages of the drug can readily be determined by those of ordinary skill in the art of medicine by assessing amelioration of the disorder or side effect in the patient, and increasing the dosage and/or frequency of treatment as desired. The optimal amount of the drug may depend upon the mode of administration, the age and the body weight of the patient, and the condition of the patient. In some embodiments, the drugs are administered at a dosage of 0.001 to 100 mg/kg of body weight of the patient; e.g., the drug is administered at a dosage of 0.01 mg to 10 mg/kg or 0.1 to 1.0 mg/kg. Preferred daily doses of the 5HT₂/5HT₃ antagonist/alpha-2 antagonist (mirtazapine) are approximately 7.5 to 200 mg/day, and preferably 15 to 45 mg/day. Preferred daily doses of setiptiline are generally from about 1 to about 50, especially about 5 to about 20 mg/day. Preferred daily doses of the SNRI (e.g. duloxetine), NSRI (e.g. milnacipran) or RIMA are approximately 1 to 200 mg/day, and preferably 20 to 120 mg/day.

It is understood that the disclosed methods are not limited to the particular methodology, protocols, and reagents described as these may vary. It is also to be understood that the terminology used herein is for the purpose of describing particular embodiments only, and is not intended to limit the scope of the present invention which will be limited only by the appended claims.

### Synergism

Both 5HT₂/5HT₃ antagonist/alpha-2 antagonists and SNRI, NSRI or RIMAs have demonstrated utility in treating a variety of conditions, such as affective disorders and pain, as discussed in more detail above. The SNRI, NSRI or RIMAs increase intrasynaptic norepinephrine by blocking the reuptake of norepinephrine by norepinephrine transporters. It has been found that inhibition of norepinephrine reuptake results in suppression of norepinephrine release. This effect is mediated through α-2 receptors, which participate in a feedback mechanism that reduces NA release as intrasynaptic NA levels increase. The α-2 antagonistic effect of a 5HT₂/5HT₃ antagonist/alpha-2 antagonist, such as mirtazapine and setiptiline, should enhance the overall effectiveness, increase the rate of onset of effectiveness, or decrease the necessary effective dose of a co-administered SNRI, NSRI or RIMA, by blocking the α-2 sites. Additionally, 5HT₂/5HT₃ antagonist/alpha-2 antagonist block the action of 5-HT at the 5HT₂ and 5HT₃ receptor sites. Thus they are anti-depressants and anti-emetics.

Both 5HT₂/5HT₃ antagonist/alpha-2 antagonists (such as mirtazapine and setiptiline) and SNRI (such as duloxetine), NSRI (e.g. milnacipran) or RIMAs have demonstrated negative side effects in the clinic. Mirtazapine induces increased appetite and weight gain as well as sedation and cognitive impairment, while milnacipran or duloxetine has been associated with nausea and insomnia. It is considered an aspect of the embodiments described herein that co-administration of a 5HT₂/5HT₃ antagonist/alpha-2 antagonist, such as mirtazapine, and a SNRI (e.g. duloxetine), NSRI (e.g. milnacipran) or RIMA, would result in reduction in the frequency or severity of one or more side effects associated with one of the two co-administered agents. In particular, it is considered an aspect of embodiments described herein that the co-administration of a 5HT₂/5HT₃ antagonist/alpha-2 antagonist, such as mirtazapine, and a SNRI (e.g. duloxetine), NSRI (e.g. milnacipran) or RIMA will result in a reduction in the sedative effects (especially the daytime sedative effects), the cognitive impairment effects, or both that are associated with mirtazapine. Additionally or alternatively, it is considered an aspect of embodiments described herein that the co-administration of a 5HT₂/5HT₃ antagonist/alpha-2 antagonist and a SNRI, NSRI or RIMA will result in a reduction in at least one side effect associated with SNRI, NSRI or RIMAs, such as nausea, insomnia or both. Such synergy may be due to positive synergistic effects, negative synergistic effects or both. In this regard, positive synergistic effects refer to the combined activity of the 5HT₂/5HT₃ antagonist/alpha-2 antagonist agent and the SNRI, NSRI or RIMA in the treatment of the target disorder such that a lower dose of each may be used; an improved side effect profile may thus be obtained due to the lower dose of each agent necessary to achieve the desired effect. Negative synergy refers to one or more synergistic effects resulting from countervailing negative side-effects of the two agents (e.g. the anti-emetic effect of the 5HT₂/5HT₃ antagonist/alpha-2 antagonist versus the nausea-inducing effect of the SNRI, NSRI or RIMA, the cognition improving effect of the SNRI, NSRI or RIMA versus the cognition impairment caused by the 5HT₂/5HT₃ antagonist/alpha-2 antagonist and/or the stimulant effect of the SNRI, NSRI or RIMA versus the sedating effect of the 5HT₂/5HT₃ antagonist/alpha-2 antagonist). Both positive and negative synergy together would imply that a lower dose of each agent could be used to achieve the desired effect and that at least one side-effect of one of the agents would be reduced below the level expected for the lower dose of that agent by a countervailing side-effect of the other agent. Thus, it is considered an aspect of embodiments described herein that in at least some embodiments, each agent may be administered at a dose lower than would be necessary to achieve a therapeutic effect if each was dosed separately, thereby giving rise to an improved side effect profile. Such improved side effect profile would include one or more of: reduced sedation (relative to normally-dosed mirtazapine or other 5HT₂/5HT₃ antagonist/alpha-2 antagonist), improved cognition (relative to normally dosed mirtazapine or other 5HT₂/5HT₃ antagonist/alpha-2 antagonist), reduced nausea (relative to normally-dosed milnacipran, duloxetine or other SNRI, NSRI or RIMA) or reduced incidence or severity of insomnia (relative to milnacipran, duloxetine or other SNRI, NSRI or RIMA). It is also considered an aspect of embodiments described herein that in at least some embodiments, the combined administration of therapeutically effective levels of mirtazapine (or other 5HT₂/5HT₃ antagonist/alpha-2 antagonist) and milnacipran or duloxetine (or other SNRI, NSRI or RIMA) will result in an improved side effect profile owing to the countervailing side-effect profiles of the two agents; such improved side effect profile would be expected to include one or more of: reduced sedation (due to the stimulating effect of milnacipran, duloxetine or other SNRI, NSRI or RIMA), improved cognition (due to the cognition-enhancing effect of milnacipran, duloxetine or other SNRI, NSRI or RIMA), reduced nausea (due to the anti-emetic effect of mirtazapine or other 5HT₂/5HT₃ antagonist/alpha-2 antagonist) or reduced incidence or severity of insomnia (due to the sedative effect of mirtazapine or other 5HT₂/5HT₃ antagonist/alpha-2 antagonist). It is also considered an aspect of embodiments described herein that in at least some embodiments both positive and negative synergy as described herein will be produced by methods and formulations according to the invention; such improved side-effect profile would include one or more of: reduced sedation greater than would be expected from merely reducing the dose or mirtazapine or other 5HT₂/5HT₃ antagonist/alpha-2 antagonist, improved cognition greater than would be expected from merely reducing the dose or mirtazapine or other 5HT₂/5HT₃ antagonist/alpha-2 antagonist, reduced nausea greater than would be expected from merely reducing the dose or milnacipran, duloxetine or other SNRI, NSRI or RIMA, or reduced incidence or severity of insomnia greater than would be expected from merely reducing the dose or milnacipran, duloxetine or other SNRI, NSRI or RIMA.

Unless defined otherwise, all technical and scientific terms used herein have the same meanings as commonly understood by one of skill in the art to which the disclosed invention belongs.

The invention may be further appreciated upon consideration of the following illustrative, non-limiting examples.

### EXAMPLE 1

### Assessing the Ability of Mirtazapine & Milnacipran or Duloxetine to Ameliorate One Another's Side Effects

In order to assess the synergistic effects on tolerability of a combination of mirtazapine and milnacipran or duloxetine, a four arm, randomized, double blind, placebo-controlled study of up to 80 normal, health subjects is conducted. The subjects receive 2 capsules per day, one in the morning and one at bedtime. Subjects are randomized into one of four equally sized study arms and receive placebo in the morning+15 mg of mirtazapine in the evening, 100 mg of milnacipran or duloxetine+15 mg of mirtazapine, 4 mg of milnacipran or duloxetine+15 mg of mirtazapine, or 100 mg of milnacipran or duloxetine+placebo. All medications are administered in an over-encapsulated format that ensures blinding of study participants, staff and investigators. All subjects are scheduled to receive a total of 6 weeks of therapy, and are required to return to the clinics after 1, 2, 4 and 6 weeks of therapy. Patients are required to complete paper self-assessments, electronic diary assessments, computer based cognitive testing, as well as vital signs and weight assessments at the clinic visits. In this trial, we confirm that mirtazapine alone does cause weight gain, sedation, and cognitive deficits and, conversely, that milnacipran or duloxetine causes insomnia, nausea/vomiting, and weight loss. We further show that combining the two drugs results in reduction in the side effects caused by either drug alone.

### EXAMPLE 1A

### Assessing the Ability of Mirtazapine & Milnacipran to Ameliorate One Another's Side Effects

In order to assess the synergistic effects on tolerability of a combination of mirtazapine and milnacipran enriched in the 1S,2R-milnacipran, a four arm, randomized, double blind, placebo-controlled study of up to 80 normal, health subjects is conducted. The subjects receive 2 capsules per day, one in the morning and one at bedtime. Subjects are randomized into one of four equally sized study arms and receive placebo in the morning+15 mg of mirtazapine in the evening, 100 mg of milnacipran having a ratio of 1S,2R-milnacipran to 1R,2S-milnacipran of about 55:45 to about 90:10 (mass/mass)+15 mg of mirtazapine, 4 mg milnacipran having a ratio of 1S,2R-milnacipran to 1R,2S-milnacipran of about 55:45 to about 90:10 (mass/mass)+15 mg of mirtazapine, or 100 mg of milnacipran having a ratio of 1S,2R-milnacipran to 1R,2S-milnacipran of about 55:45 to about 90:10 (mass/mass)+placebo. All medications are administered in an over-encapsulated format that ensures blinding of study participants, staff and investigators. All subjects are scheduled to receive a total of 6 weeks of therapy, and are required to return to the clinics after 1, 2, 4 and 6 weeks of therapy. Patients are required to complete paper self-assessments, electronic diary assessments, computer based cognitive testing, as well as vital signs and weight assessments at the clinic visits. In this trial, we confirm that mirtazapine alone does cause weight gain, sedation, and cognitive deficits and, conversely, that milnacipran or duloxetine causes insomnia, nausea/vomiting, and weight loss. We further show that combining the two drugs results in reduction in the side effects caused by either drug alone.

### EXAMPLE 2

### Demonstrating Synergy in the Treatment of Depression

In order to assess the synergistic effects on efficacy of a combination of mirtazapine and milnacipran or duloxetine, a four arm, randomized, double blind, placebo-controlled study of up to 100 patients suffering from major depressive episode (see DSM IV) is conducted. The subjects receive 2 capsules per day, one in the morning and one at bedtime. The dose of milnacipran or duloxetine used is determined from the study described in Example 1; all such doses are typically considered to be ineffective. Subjects are randomized into one of five equally sized study arms, and receive either placebo in the morning and in the evening either (1) placebo; (2) placebo+30 mg of mirtazapine+25 mg milnacipran or 20 mg duloxetine; (3) placebo+7.5 mg of mirtazapine+25 mg of milnacipran or 20 mg duloxetine; (4) placebo+100 mg of mirtazapine or 40 mg duloxetine; or (5) placebo+30 mg of mirtazapine. Note that 7.5 and 15 mg/day doses of mirtazapine, taken in isolation, are typically considered to be ineffective doses of mirtazapine. All medications are administered in an over-encapsulated format that ensures blinding of study participants, staff and investigators. All subjects are scheduled to receive a total of 8 weeks of therapy, and are required to return to the clinics after 1, 2, 4 and 8 weeks of therapy. Patients are required to complete paper self-assessments, electronic diary assessments, computer based cognitive testing, as well as vital signs and weight assessments at the clinic visits. In particular, patients' response on the HAM-D, Beck Depression Inventory, and HAM-A are assessed. In this trial, we confirm that low dose mirtazapine+low dose milnacipran or duloxetine is more effective in treating depression than 30 mg of mirtazapine/day.

### EXAMPLE 2A

### Demonstrating Synergy in the Treatment of Depression with Enantiomerically Enriched Milnacipran

In order to assess the synergistic effects on efficacy of a combination of mirtazapine and milnacipran having a ratio of 1S,2R-milnacipran to 1R,2S-milnacipran of about 65:45 to about 90:10 (mass/mass), a four arm, randomized, double blind, placebo-controlled study of up to 100 patients suffering from major depressive episode (see DSM IV) is conducted. The subjects receive 2 capsules per day, one in the morning and one at bedtime. The dose of milnacipran or duloxetine used is determined from the study described in Example 1; all such doses are typically considered to be ineffective. Subjects are randomized into one of five equally sized study arms, and receive either placebo in the morning and in the evening either (1) placebo; (2) placebo+30 mg of mirtazapine+25 mg milnacipran having a ratio of 1S,2R-milnacipran to 1R,2S-milnacipran of about 55:45 to about 90:10 (mass/mass); (3) placebo+7.5 mg of mirtazapine+25 mg of milnacipran having a ratio of 1 S,2R-milnacipran to 1R,2S-milnacipran of about 55:45 to about 90:10 (mass/mass); (4) placebo+100 mg of mirtazapine or 20 mg racemic milnacipran (1:1 ratio (mass/mass) 1S,2R- to 1R,2S-enantiomer); or (5) placebo+30 mg of mirtazapine. Note that 7.5 and 15 mg/day doses of mirtazapine, taken in isolation, are typically considered to be ineffective doses of mirtazapine. All medications are administered in an over-encapsulated format that ensures blinding of study participants, staff and investigators. All subjects are scheduled to receive a total of 8 weeks of therapy, and are required to return to the clinics after 1, 2, 4 and 8 weeks of therapy. Patients are required to complete paper self-assessments, electronic diary assessments, computer based cognitive testing, as well as vital signs and weight assessments at the clinic visits.

### EXAMPLE 3

### Demonstrating Synergy in the Treatment of Neuropathic Pain

In order to assess the synergistic effects on efficacy of a combination of mirtazapine and milnacipran or duloxetine, a four arm, randomized, double blind, placebo-controlled study of up to 100 patients suffering from neuropathic pain (from diabetic neuropathy and/or postherpetic neuralgia) is conducted. The subjects receive 2 capsules per day, one in the morning and one at bedtime. The dose of milnacipran or duloxetine used is determined from the study described in Example 1; all such doses are typically considered to be ineffective when taken by themselves. Subjects are randomized into one of five equally sized study arms, and receive either placebo in the morning and in the evening either (1) placebo; (2) placebo+30 mg of mirtazapine+25 mg milnacipran or 20 mg duloxetine; (3) placebo+7.5 mg of mirtazapine+25 mg of milnacipran or 20 mg duloxetine; (4) placebo+100 mg of mirtazapine or 40 mg duloxetine; or (5) placebo+30 mg of mirtazapine. Note that 7.5 and 15 mg/day doses of mirtazapine, taken in isolation, are typically considered to be ineffective doses of mirtazapine. All medications are administered in an over-encapsulated format that ensures blinding of study participants, staff and investigators. All subjects are scheduled to receive a total of 8 weeks of therapy, and are required to return to the clinics after 1, 2, 4 and 8 weeks of therapy. Patients are required to complete paper self-assessments, electronic diary assessments, computer based cognitive testing, as well as vital signs and weight assessments at the clinic visits.

The Patient Experience Diary (PED)

Improvement in patient pain is an essential feature of any efficacious therapeutic intervention for neuropathic pain. Advances in both the technology and methodology of real-time data collection have enabled researchers to capture reliable and valid momentary data from subjects in the real world. [Stone A, S. S., Schwartz J, Broderick J, Hufford M, Patient compliance with paper and electronic diaries. Control Clin. Trials, 2003, 24(3), 182-99.] In this study, subjects are asked to provide information at up to five different times during the course of the day, including a morning report, evening report and on average, three random daily pain prompts.

To facilitate accurate and timely assessments of pain, an electronic diary system has been implemented for this study: Patient Experience Diary or PED (invivodata, inc., Pittsburgh, Pa.). The PED uses invivodata's proprietary software loaded on a personal digital assistant (PDA). The core of the PED data is the collection of subject self-reported data. In this study, the data are collected via entries made by subjects at relevant times into the PED. Specifically, the PED software enables subjects' pain assessments to be completed at a variety of times throughout the day, as required by the protocol.

The PED permits the collection of real-time, self-reported pain data by random report prompting multiple times daily, and also asks individuals to recall daily pain and weekly pain during the corresponding daily and weekly reports. The following table highlights key assessments implemented on the PED:

| Study Phase(s) | Study Need | DIARY FUNCTION |
|---|---|---|
| Baseline and | Daily and “real- | Morning report |
| Treatment | time” pain data and | Random prompts |
| | daily mood data | Evening report |
| | Weekly | Weekly report |
| | retrospective pain | |
| | and QOL data | |
| | Confirm medication | Self-initiated study medication |
| | administration | |

The primary pain outcome variable is measured on the electronic diary. There are several additional pieces of pain information that are collected routinely during the clinic visits scheduled for Baseline Study Visit (BL2/T×0), T×6, and T×12, such as pain self-report, psychophysical testing variables and standardized tenderness measures. Visual analog scale-based pain measurements are captured on a dedicated, daily and weekly pain recall case report form at study visits. These alternative pain assessment scales are evaluated as secondary variables, but do not substitute for data collected on the electronic diary.

In addition to pain ratings, assessments of mood and appetite are also recorded using the electronic diary. Subjects rate their mood and sedation nightly using a visual analog scale based on the Bond-Lader mood scale. [Bond, A.a.L., M., The use of analogue scales in rating subjective feelings. British Journal of Medical Psychology, 1974, 47, 211-218.] Subjects rate appetite on a weekly basis using a “drop-down” menu with the following choices: “increased”, “decreased”, or “no change”.

Training and Participant Usage. Following both a didactic and interactive training session at the Screening visit, participants are asked to use the PED to record symptoms daily for the duration of the 14-week study. The PED in this study prompts participants for several different types of input. In the morning, when participants first wake up, they report on their current pain level and their pain over the previous 24 hours. On multiple occasions throughout the day, random prompts requesting information about current level of pain are presented. Finally, at bedtime, another series of questions is presented, including a passive check of medication compliance. And on every 7th evening, participants are presented with the weekly report, which triggers another specific set of questions regarding their recall of pain and fatigue for the week. Each of these series of questions is designed to be easy and quick to complete, as minimizing burden on the participants has been carefully considered. All questions presented at all prompts are listed in the invivodata study coordinator manual.

To avoid interruptions to daily life, the random prompts may be suspended or delayed as needed for a period of 30 minutes up to 2 hours. The PED are pre-programmed with a standard wake period and evening report period, both substantial in duration to account for individual variations and habits. Following evening report, subjects place the PED in its dedicated modem for overnight data uploading and then awakening or activating PED the following morning within the programmed wake period. Because patient compliance is one of the major reasons to use the PED approach as compared to paper diaries, the electronic diary data are electronically time and date stamped when entries are made. There is no provision for the participant to make late entries.

Brief Pain Inventory: Clinical pain is also assessed using the Brief Pain Inventory (BPI). The BPI is a short, self-report measure that was originally developed for use in cancer patients to assess pain intensity and the impact of pain on the patient's life. [Tan G, J. M., Thornby J I, Shanti B F, Validation of the Brief Pain Inventory for chronic nonmalignant pain, J. Pain, 2004, 5(2) (March), 133-7; Keller S, B. C., Dodd S L, Schein J, Mendoza T R, Cleeland C S, Validity of the brief pain inventory for use in documenting the outcomes of patients with non-cancer pain, Clin. J. Pain, 2004, 20(5) (September), p. 309-18.] Recently, the BPI was validated for use in chronic, nonmalignant pain such as low back pain and arthritis with reliability and validity comparable to reports from the cancer literature and with internal consistency to support using the BPI as an outcome variable in treatment outcome studies. Tan, 2004. Patients are asked to rate their current pain intensity as well as their worst, least and average pain in the last 24 hours on a 0-10 rating scale (0=“no pain” and 10=“pain as bad as you can imagine”). Additionally, patients are asked to rate the extent that pain interferes with their life across 7 domains: general activity, walking, mood, sleep, work, relations with other persons, and enjoyment of life; interference is also rated on a 0-10 scale (0=“does not interfere” and 10=“completely interferes”).

In this trial, we confirm that low dose mirtazapine+low dose milnacipran or duloxetine is more effective in treating neuropathic pain than 30 mg of mirtazapine/day.

### Clinical Endpoints

Efficacy of the combination of the combination of mirtazapine and milnacipran or duloxetine is assessed using the following methods:

Self-reporting questionnaires

Electronic patient experience diary (PED)

Electronic cognitive testing.

### EXAMPLE 3A

### Demonstrating Synergy in the Treatment of Neuropathic Pain

In order to assess the synergistic effects on efficacy of a combination of mirtazapine and enantiomerically enriched milnacipran, a four arm, randomized, double blind, placebo-controlled study of up to 100 patients suffering from neuropathic pain (from diabetic neuropathy and/or postherpetic neuralgia) is conducted. The study is conducted essentially as set forth in Example 3, above, except that the arms employing milnacipran instead employ milnacipran in which the ratio of 1S,2R-milnacipran to 1R,2S-milnacipran is in the range of about 55:55 to about 90:10 (mass/mass) and the arms employing duloxetine instead employ racemic milnacipran (1:1 ratio of 1S,2R- and 1R,2S-enantiomers).

### EXAMPLE 4

### Assessing the Ability of Mirtazapine and Milnacipran to Ameliorate One Another's Side Effects

In order to assess the synergistic effects on tolerability of a combination of mirtazapine and milnacipran, a four arm, randomized, double blind, placebo-controlled study of up to 80 normal, health subjects is conducted. The subjects receive 2 capsules per day, one in the morning and one at bedtime. Subjects are randomized into one of four equally sized study arms and receive placebo in the morning and in the evening either: (1) 15 mg of mirtazapine; (2) 50 mg milnacipran; (3) 15 mg of mirtazapine and 100 mg of milnacipran; or (4) 15 mg of mirtazapine and 50 mg milnacipran. All medications are administered in an over-encapsulated format that ensures blinding of study participants, staff and investigators. All subjects are scheduled to receive a total of 6 weeks of therapy, and are required to return to the clinics after 1, 2, 4 and 6 weeks of therapy. Patients are required to complete paper self-assessments, electronic diary assessments, computer based cognitive testing, as well as vital signs and weight assessments at the clinic visits. In this trial, we confirm that mirtazapine alone does cause weight gain, sedation, and cognitive deficits and, conversely, that milnacipran or duloxetine causes insomnia, nausea/vomiting, and weight loss. We further show that combining the two drugs results in reduction in the side effects caused by either drug alone.

### EXAMPLE 4A

### Assessing the Ability of Mirtazapine and Enantiomerically Enriched Milnacipran to Ameliorate One Another's Side Effects

In order to assess the synergistic effects on tolerability of a combination of mirtazapine and milnacipran, a four arm, randomized, double blind, placebo-controlled study of up to 80 normal, health subjects is conducted. The subjects receive 2 capsules per day, one in the morning and one at bedtime. Subjects are randomized into one of four equally sized study arms and receive placebo in the morning and in the evening either: (1) 15 mg of mirtazapine; (2) 50 mg enantiomerically enriched milnacipran (1S,2R- to 2R, 1S-milnacipran of about 55:45 to about 90:10 (mass/mass); (3) 15 mg of mirtazapine and 100 mg of enantiomerically enriched milnacipran (1S,2R- to 2R,1S-milnacipran of about 55:45 to about 90:10 (mass/mass); or (4) 15 mg of mirtazapine and 50 mg enantiomerically enriched milnacipran (1S,2R- to 2R,1S-milnacipran of about 55:45 to about 90:10 (mass/mass). Except for the substitution of enantiomerically enriched milnacipran (1S,2R- to 2R,1S-milnacipran of about 55:45 to about 90:10 (mass/mass)) for racemic milnacipran (ratio of 1S,2R- to 2R,1S-milnacipran of 1:1 (mass/mass)), this study is carried out essentially as outlined in Example 4, above.

### EXAMPLE 5

### Assessing the Ability of Mirtazapine and Duloxetine to Ameliorate One Another's Side Effects

In order to assess the synergistic effects on tolerability of a combination of mirtazapine and duloxetine, a four arm, randomized, double blind, placebo-controlled study of up to 80 normal, health subjects is conducted. The subjects receive 2 capsules per day, one in the morning and one at bedtime. Subjects are randomized into one of four equally sized study arms and receive placebo in the morning and in the evening either: (1) 15 mg of mirtazapine; (2) 30 mg duloxetine; (3) 15 mg of mirtazapine and 60 mg of duloxetine; or (4) 15 mg of mirtazapine 30 mg duloxetine. All medications are administered in an over-encapsulated format that ensures blinding of study participants, staff and investigators. All subjects are scheduled to receive a total of 6 weeks of therapy, and are required to return to the clinics after 1, 2, 4 and 6 weeks of therapy. Patients are required to complete paper self-assessments, electronic diary assessments, computer based cognitive testing, as well as vital signs and weight assessments at the clinic visits. In this trial, we confirm that mirtazapine alone does cause weight gain, sedation, and cognitive deficits and, conversely, that duloxetine causes insomnia, nausea/vomiting, and weight loss. We further show that combining the two drugs results in reduction in the side effects caused by either drug alone.

### EXAMPLE 6

### Demonstrating Synergy in the Treatment of Depression

In order to assess the synergistic effects on efficacy of a combination of mirtazapine and milnacipran, a four arm, randomized, double blind, placebo-controlled study of up to 100 patients suffering from major depressive episode (see DSM IV) is conducted. The subjects receive 2 capsules per day, one in the morning and one at bedtime. The dose of milnacipran used is determined from the study described in Example 1; all such doses are typically considered to be ineffective. Subjects are randomized into one of five equally sized study arms, and receive placebo in the morning and in the evening either: (1) placebo+30 mg of mirtazapine; (2) 50 mg milnacipran+7.5 mg of mirtazapine; (3) 100 mg milnacipran+15 mg of mirtazapine; or (4) 100 mg milnacipran+30 mg of mirtazapine. Note that 7.5 and 15 mg/day doses of mirtazapine, taken in isolation, are typically considered to be ineffective doses of mirtazapine. All medications are administered in an over-encapsulated format that ensures blinding of study participants, staff and investigators. All subjects are scheduled to receive a total of 8 weeks of therapy, and are required to return to the clinics after 1, 2, 4 and 8 weeks of therapy. Patients are required to complete paper self-assessments, electronic diary assessments, computer based cognitive testing, as well as vital signs and weight assessments at the clinic visits. In particular, patients' response on the HAM-D, Beck Depression Inventory, and HAM-A are assessed. In this trial, we confirm that low dose mirtazapine+low dose milnacipran is more effective in treating depression than 30 mg of mirtazapine/day.

### EXAMPLE 6A

### Demonstrating Synergy in the Treatment of Depression

In order to assess the synergistic effects on efficacy of a combination of mirtazapine and milnacipran, a four arm, randomized, double blind, placebo-controlled study of up to 100 patients suffering from major depressive episode (see DSM IV) is conducted. The subjects receive 2 capsules per day, one in the morning and one at bedtime. The dose of milnacipran used is determined from the study described in Example 1A; all such doses are typically considered to be ineffective. Subjects are randomized into one of five equally sized study arms, and receive placebo in the morning and in the evening either: (1) placebo+30 mg of mirtazapine; (2) 50 mg milnacipran having a ratio of 1S,2R-milnacipran to 1R,2S-milnacipran of about 55:45 to about 90:10 (mass/mass)+7.5 mg of mirtazapine; (3) 100 mg milnacipran having a ratio of 1S,2R-milnacipran to 1R,2S-milnacipran of about 55:45 to about 90:10 (mass/mass)+15 mg of mirtazapine; or (4) 100 mg milnacipran having a ratio of 1S,2R-milnacipran to 1R,2S-milnacipran of about 55:45 to about 90:10 (mass/mass)+30 mg of mirtazapine. Aside from substituting milnacipran having a ratio of 1S,2R-milnacipran to 1R,2S-milnacipran of about 55:45 to about 90:10 (mass/mass) for racemic milnacipran (having a ratio of 1S,2R-milnacipran to 1R,2S-milnacipran of about 1:1 (mass/mass)), this study is carried out essentially as outlined in Example 6, above.

### EXAMPLE 7

### Demonstrating Synergy in the Treatment of Neuropathic Pain

In order to assess the synergistic effects on efficacy of a combination of mirtazapine and milnacipran, a four arm, randomized, double blind, placebo-controlled study of up to 100 patients suffering from neuropathic pain (from diabetic neuropathy and/or postherpetic neuralgia) is conducted. The subjects receive 2 capsules per day, one in the morning and one at bedtime. The dose of milnacipran used is determined from the study described in Example 1; all such doses are typically considered to be ineffective. Subjects are randomized into one of five equally sized study arms, and receive placebo in the morning and in the evening either: (1) placebo+30 mg of mirtazapine; (2) 50 mg milnacipran+7.5 mg of mirtazapine; (3) 100 mg milnacipran+15 mg of mirtazapine; or (4) 100 mg milnacipran+30 mg of mirtazapine. Note that 7.5 and 15 mg/day doses of mirtazapine, taken in isolation, are typically considered to be ineffective doses of mirtazapine. All medications are administered in an over-encapsulated format that ensures blinding of study participants, staff and investigators. All subjects are scheduled to receive a total of 8 weeks of therapy, and are required to return to the clinics after 1, 2, 4 and 8 weeks of therapy. Patients are required to complete paper self-assessments, electronic diary assessments, computer based cognitive testing, as well as vital signs and weight assessments at the clinic visits.

The Patient Experience Diary (PED)

Clearly, improvement in patient pain is an essential feature of any efficacious therapeutic intervention for neuropathic pain. Advances in both the technology and methodology of real-time data collection have enabled researchers to capture reliable and valid momentary data from subjects in the real world. [Stone A, S. S., Schwartz J, Broderick J, Hufford M, Patient compliance with paper and electronic diaries. Control Clin. Trials, 2003, 24(3), 182-99.] In this study, subjects are asked to provide information at up to five different times during the course of the day, including a morning report, evening report and on average, three random daily pain prompts.

To facilitate accurate and timely assessments of pain, an electronic diary system has been implemented for this study: Patient Experience Diary or PED (invivodata, inc., Pittsburgh, Pa.). The PED uses invivodata's proprietary software loaded on a personal digital assistant (PDA). The core of the PED data is the collection of subject self-reported data. In this study, the data are collected via entries made by subjects at relevant times into the PED. Specifically, the PED software enables subjects' pain assessments to be completed at a variety of times throughout the day, as required by the protocol.

The PED permits the collection of real-time, self-reported pain data by random report prompting multiple times daily, and also asks individuals to recall daily pain and weekly pain during the corresponding daily and weekly reports. The following table highlights key assessments implemented on the PED:

| Study Phase(s) | Study Need | DIARY FUNCTION |
|---|---|---|
| Baseline and | Daily and “real- | Morning report |
| Treatment | time” pain data and | Random prompts |
| | daily mood data | Evening report |
| | Weekly | Weekly report |
| | retrospective pain | |
| | and QOL data | |
| | Confirm medication | Self-initiated study medication |
| | administration | |

The primary pain outcome variable is measured on the electronic diary. There are several additional pieces of pain information that are collected routinely during the clinic visits scheduled for Baseline Study Visit (BL2/T×0), T×6, and T×12, such as pain self-report, psychophysical testing variables and standardized tenderness measures. Visual analog scale-based pain measurements are captured on a dedicated, daily and weekly pain recall case report form at study visits. These alternative pain assessment scales are evaluated as secondary variables, but do not substitute for data collected on the electronic diary.

In addition to pain ratings, assessments of mood and appetite are also recorded using the electronic diary. Subjects rate their mood and sedation nightly using a visual analog scale based on the Bond-Lader mood scale. [Bond, A.a.L., M., The use of analogue scales in rating subjective feelings. British Journal of Medical Psychology, 1974, 47, 211-218.] Subjects rate appetite on a weekly basis using a “drop-down” menu with the following choices: “increased”, “decreased”, or “no change”.

Training and Participant Usage. Following both a didactic and interactive training session at the Screening visit, participants are asked to use the PED to record symptoms daily for the duration of the 14-week study. The PED in this study prompts participants for several different types of input. In the morning, when participants first wake up, they report on their current pain level and their pain over the previous 24 hours. On multiple occasions throughout the day, random prompts requesting information about current level of pain are presented. Finally, at bedtime, another series of questions is presented, including a passive check of medication compliance. And on every 7th evening, participants are presented with the weekly report, which triggers another specific set of questions regarding their recall of pain and fatigue for the week. Each of these series of questions is designed to be easy and quick to complete, as minimizing burden on the participants has been carefully considered. All questions presented at all prompts are listed in the invivodata study coordinator manual.

To avoid interruptions to daily life, the random prompts may be suspended or delayed as needed for a period of 30 minutes up to 2 hours. The PED are pre-programmed with a standard wake period and evening report period, both substantial in duration to account for individual variations and habits. Following evening report, subjects place the PED in its dedicated modem for overnight data uploading and then awakening or activating PED the following morning within the programmed wake period. Because patient compliance is one of the major reasons to use the PED approach as compared to paper diaries, the electronic diary data are electronically time and date stamped when entries are made. There is no provision for the participant to make late entries.

Brief Pain Inventory: Clinical pain is also assessed using the Brief Pain Inventory (BPI). The BPI is a short, self-report measure that was originally developed for use in cancer patients to assess pain intensity and the impact of pain on the patient's life. [Tan G, J. M., Thornby J I, Shanti B F, Validation of the Brief Pain Inventory for chronic nonmalignant pain, J. Pain, 2004, 5(2) (March), 133-7; Keller S, B. C., Dodd S L, Schein J, Mendoza T R, Cleeland C S, Validity of the brief pain inventory for use in documenting the outcomes of patients with non-cancer pain, Clin. J. Pain, 2004, 20(5) (September), p. 309-18.] Recently, the BPI was validated for use in chronic, nonmalignant pain such as low back pain and arthritis with reliability and validity comparable to reports from the cancer literature and with internal consistency to support using the BPI as an outcome variable in treatment outcome studies. Tan, 2004. Patients are asked to rate their current pain intensity as well as their worst, least and average pain in the last 24 hours on a 0-10 rating scale (0=“no pain” and 10=“pain as bad as you can imagine”). Additionally, patients are asked to rate the extent that pain interferes with their life across 7 domains: general activity, walking, mood, sleep, work, relations with other persons, and enjoyment of life; interference is also rated on a 0-10 scale (0=“does not interfere” and 10=“completely interferes”).

In this trial, we confirm that low dose mirtazapine+low dose milnacipran is more effective in treating neuropathic pain than 30 mg of mirtazapine/day.

Clinical Endpoints

Efficacy of the combination of the combination of mirtazapine and milnacipran is assessed using the following methods:

Self-reporting questionnaires

Electronic patient experience diary (PED)

Electronic cognitive testing.

### EXAMPLE 7A

### Demonstrating Synergy in the Treatment of Neuropathic Pain

In order to assess the synergistic effects on efficacy of a combination of mirtazapine and milnacipran having a ratio of 1S,2R-milnacipran to 1R,2S-milnacipran of about 55:45 to about 90:10 (mass/mass), a four arm, randomized, double blind, placebo-controlled study of up to 100 patients suffering from neuropathic pain (from diabetic neuropathy and/or postherpetic neuralgia) is conducted. The subjects receive 2 capsules per day, one in the morning and one at bedtime. The dose of milnacipran having a ratio of 1S,2R-milnacipran to 1R,2S-milnacipran of about 55:45 to about 90:10 (mass/mass) used in this study is determined from the study described in Example 1A; all such doses are typically considered to be ineffective. Subjects are randomized into one of five equally sized study arms, and receive placebo in the morning and in the evening either: (1) placebo+30 mg of mirtazapine; (2) 50 mg milnacipran having a ratio of 1S,2R-milnacipran to 1R,2S-milnacipran of about 55:45 to about 90:10 (mass/mass)+7.5 mg of mirtazapine; (3) 100 mg milnacipran having a ratio of 1S,2R-milnacipran to 1R,2S-milnacipran of about 55:45 to about 90:10 (mass/mass)+15 mg of mirtazapine; or (4) 100 mg milnacipran having a ratio of 1S,2R-milnacipran to 1R,2S-milnacipran of about 55:45 to about 90:10 (mass/mass)+30 mg of mirtazapine. Aside from substituting racemic mirtazapine (having a ratio of 1S,2R-milnacipran to 1R,2S-milnacipran is about 1:1) with milnacipran having a ratio of 1S,2R-milnacipran to 1R,2S-milnacipran of about 55:45 to about 90:10 (mass/mass), the procedure used in this study is essentially as set forth in Example 7.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments provided herein described herein may be employed in practicing the invention. It is intended that the following claims define the scope provided herein and that methods and structures within the scope of these claims and their equivalents be covered thereby.

What is claimed is:

1. A method of reducing the incidence or severity of one or more side effects associated with administration of at least a first therapeutic agent, a second therapeutic agent or both in the treatment of a disorder in a patient, wherein the first therapeutic agent has 5HT₂/5HT₃ antagonist and alpha-2 antagonist activity, and the second therapeutic agent possesses both serotonin reuptake inhibitory activity and norepinephrine reuptake inhibitory activity or is a reversible inhibitor of monoamine oxidase A (RIMA), comprising administering to the patient an effective amount of the first therapeutic agent and the second therapeutic agent.

2. The method of claim 1, wherein at least one side effect that is reduced is daytime sedation, cognitive impairment, iatrogenic weight gain, nausea or emesis.

3. The method of claim 1, wherein the first therapeutic agent comprises one or members of the group consisting of mirtazapine, mirtazapine enriched in either the R- or S-enantiomer, setiptiline or a combination thereof.

4. The method of claim 3, wherein the first therapeutic agent comprises mirtazapine or mirtazapine enriched in either the R- or S-enantiomer.

5. The method of claim 3, wherein the first therapeutic agent comprises setiptiline.

6. The method of claim 1, wherein when the second therapeutic agent possesses serotonin reuptake inhibitory activity and norepinephrine reuptake inhibitory activity; and the ratio of serotonin reuptake inhibitory activity to norepinephrine reuptake inhibitory activity is in the range of about 1:50 to 50:1, about 1:20 to 20:1 or about 1:10 to 10:1.

7. The method of claim 1, wherein the second therapeutic agent comprises a serotonin norepinephrine reuptake inhibitor (SNRI).

8. The method of claim 7, wherein the SNRI is selected from the group consisting of duloxetine, venlafaxine, desvenlafaxine, indeloxazine and pharmaceutically acceptable salts, derivatives and combinations thereof.

9. The method of claim 1, wherein the second therapeutic agent comprises a norepinephrine serotonin reuptake inhibitor (NSRI).

10. The method of claim 9, wherein the NSRI is selected from the group consisting of milnacipran, derivatives of milnacipran, bicifadine, and pharmaceutically acceptable salts, derivatives and combinations thereof.

11. The method of claim 10, wherein the NSRI is a racemic mixture of 1S,2R-milnacipran and 1R,2S-milnacipran or is milnacipran enriched in the 1S,2R-enantiomer of milnacipran, wherein a mass/mass ratio of the (1S,2R) enantiomer to the (1R,2S) enantiomer of milnacipran is greater than 1:1, greater than or equal to about 55:45, greater than or equal to about 60:40, greater than or equal to about 65:35, greater than or equal to about 70:30, greater than or equal to about 75:25, greater than or equal to about 80:20, greater than or equal to about 82:18, greater than or equal to about 84:16, greater than or equal to about 86:14, greater than or equal to about 88:12, greater than or equal to about 90:10, greater than or equal to about 91:9, greater than or equal to about 92:8, greater than or equal to about 93:7, greater than or equal to about 94:6, greater than or equal to about 95:5, greater than or equal to about 96:4, greater than or equal to about 97:3, greater than or equal to about 98:2, greater than or equal to about 99:1, greater than or equal to about 99.5:0.5, in a range of about 55:45 to about 95:5, in a range of about 55:45 to about 92.5:7.5, in a range of about 55:45 to about 90:10, in a range of about 60:40 to about 95:5, in a range of about 60:40 to about 92.5:7.5, in a range of about 60:40 to about 90:10, in a range of about 65:35 to about 95:5, in a range of about 65:35 to about 92.5:7.5, in a range of about 65:35 to about 90:10, in a range of about 60:30 to about 95:5, in a range of about 70:30 to about 92.5:7.5, in a range of about 70:30 to about 90:10.

12. The method of claim 1, wherein the second therapeutic agent comprises a reversible inhibitor of monoamine oxidase A (RIMA).

13. The method claim 12, wherein the second therapeutic agent comprises a RIMA selected from the group consisting of brofaromine, moclobemide, and pharmaceutically acceptable salts, derivatives and combinations thereof.

14. The method of claim 1, wherein the first and second therapeutic agents are administered as a unit dose.

15. The method of claim 14, wherein the unit dose provides immediate release of at least a portion of the first therapeutic agent.

16. The method of claim 15, wherein the unit dose provides immediate release of substantially all of the first therapeutic agent.

17. The method of claim 15, wherein the unit dose provides delayed release of at least a portion of the second therapeutic agent.

18. The method of claim 17, wherein the unit dose provides delayed release of substantially all of the second therapeutic agent.

19. The method of claim 14, wherein the unit dose is administered to the patient within about 4 hours before bed.

20. The method of claim 19, wherein the unit dose is administered to the patient within about 2 hours before bed, within about 1 hour before bed or substantially immediately before bed.

21. The method of claim 14, wherein the unit dose provides immediate release of at least a portion of the second therapeutic agent.

22. The method of claim 21, wherein the unit dose provides immediate release of substantially all of the second therapeutic agent.

23. The method of claim 21, wherein the unit dose provides delayed release of at least a portion of the first therapeutic agent.

24. The method of claim 21, wherein the unit dose provides delayed release of substantially all of the first therapeutic agent.

25. The method of claim 21, wherein the unit dose is administered to the patient within about 4 hours after waking.

26. The method of claim 25, wherein the unit dose is administered to the patient within about 2 hours after waking.

27. The method of claim 1, wherein the first therapeutic agent is administered before bed and the second therapeutic agent is administered after waking.

28. The method of claim 27, wherein the first therapeutic agent is administered within about 4 hours before bed.

29. The method of claim 28, wherein the first therapeutic agent is administered within about 2 hours before bed.

30. The method of claim 29, wherein the first therapeutic agent is administered within about 1 hour before bed.

31. The method of claim 30, wherein the first therapeutic agent is administered substantially immediately before bed.

32. The method of claim 27, wherein the second therapeutic agent is administered within about 4 hours of waking.

33. The method of claim 32, wherein the second therapeutic agent is administered within about 2 hours after waking.

34. The method of claim 33, wherein the second therapeutic agent is administered within about 1 hour after waking or before, with or after a meal.

35. The method of claim 1, wherein at least one side effect that is reduced is daytime sedation, cognitive impairment, iatrogenic weight gain, nausea or emesis.

36. The method of claim 35, wherein the method provides reduction in two or more side effects selected from the group consisting of daytime sedation, cognitive impairment, iatrogenic weight gain, nausea and emesis.

37. The method of claim 1, wherein the disorder is selected from the group consisting of depression, schizophrenia, anxiety disorders, affective disorders, sleep-related breathing disorders, insomnia, migraine headache, chronic tension-type headache, hot flashes, chronic lower back pain, neuropathic pain (e.g. from diabetic peripheral neuropathy) and functional somatic syndromes.

38. The method of claim 37, wherein the disorder is an anxiety disorder selected from the group consisting of generalized anxiety disorder, panic disorder, phobias, and post-traumatic stress disorder.

39. The method of claim 37, wherein the disorder is a sleep-related breathing disorder selected from the group consisting of sleep apnea, sleep hypopnea, upper airway resistance syndrome, and snoring.

40. The method of claim 37, wherein the disorder is a functional somatic syndrome selected from the group consisting of fibromyalgia syndrome, chronic fatigue syndrome, and irritable bowel syndrome.

41. A formulation comprising an effective amount of a combination of a first therapeutic agent and a second therapeutic agent, wherein the first therapeutic agent is a 5HT₂/5HT₃ antagonist/alpha-2 antagonist and the second therapeutic agent possesses both serotonin reuptake inhibitory activity and norepinephrine reuptake inhibitory activity or is a reversible inhibitor of monoamine oxidase A (RIMA).

42. The formulation of claim 41, wherein the first therapeutic agent comprises one or members of the group consisting of mirtazapine, which may optionally be enriched in either the R- or S-enantiomer, setiptiline or a combination thereof.

43. The formulation of claim 42, wherein the first therapeutic agent comprises mirtazapine, which may optionally be enriched in either the R- or S-enantiomer.

44. The formulation of claim 42, wherein the first therapeutic agent comprises setiptiline.

45. The formulation of claim 42, wherein when the second therapeutic agent possesses serotonin reuptake inhibitory activity and norepinephrine reuptake inhibitory activity; and the ratio of serotonin reuptake inhibitory activity to norepinephrine reuptake inhibitory activity is in the range of about 1:50 to 50:1, about 1:20 to 20:1 or about 1:10 to 10:1.

46. The formulation of claim 41, wherein the second therapeutic agent comprises a SNRI, NSRI or RIMA (SNRI).

47. The formulation of claim 46, wherein the SNRI is selected from the group consisting of duloxetine, venlafaxine, desvenlafaxine, indeloxazine and pharmaceutically acceptable salts, derivatives and combinations thereof.

48. The formulation of claim 41, wherein the second therapeutic agent comprises a NSRI.

49. The formulation of claim 48, wherein the NSRI is selected from the group consisting of milnacipran, derivatives of milnacipran, bicifadine, and pharmaceutically acceptable salts, derivatives and combinations thereof.

50. The method of claim 49, wherein the NSRI is a racemic mixture of 1S,2R-milnacipran and 1R,2S-milnacipran or is milnacipran enriched in the 1S,2R-enantiomer of milnacipran, wherein a mass/mass ratio of the (1S,2R) enantiomer to the (1R,2S) enantiomer of milnacipran is greater than 1:1, greater than or equal to about 55:45, greater than or equal to about 60:40, greater than or equal to about 65:35, greater than or equal to about 70:30, greater than or equal to about 75:25, greater than or equal to about 80:20, greater than or equal to about 82:18, greater than or equal to about 84:16, greater than or equal to about 86:14, greater than or equal to about 88:12, greater than or equal to about 90:10, greater than or equal to about 91:9, greater than or equal to about 92:8, greater than or equal to about 93:7, greater than or equal to about 94:6, greater than or equal to about 95:5, greater than or equal to about 96:4, greater than or equal to about 97:3, greater than or equal to about 98:2, greater than or equal to about 99:1, greater than or equal to about 99.5:0.5, in a range of about 55:45 to about 95:5, in a range of about 55:45 to about 92.5:7.5, in a range of about 55:45 to about 90:10, in a range of about 60:40 to about 95:5, in a range of about 60:40 to about 92.5:7.5, in a range of about 60:40 to about 90:10, in a range of about 65:35 to about 95:5, in a range of about 65:35 to about 92.5:7.5, in a range of about 65:35 to about 90:10, in a range of about 60:30 to about 95:5, in a range of about 70:30 to about 92.5:7.5, in a range of about 70:30 to about 90:10.

51. The formulation of claim 41, wherein the second therapeutic agent comprises a reversible inhibitor of monoamine oxidase A (RIMA).

52. The formulation of claim 51, wherein the RIMA is selected from the group consisting of brofaromine, moclobemide, and pharmaceutically acceptable salts, derivatives and combinations thereof.

53. The formulation of claim 41, wherein the first and second therapeutic agents are administered in a unit dose.

54. The formulation of claim 53, wherein the unit dose provides immediate release of at least a portion of the first therapeutic agent.

55. The formulation of claim 54, wherein the unit dose provides immediate release of substantially all of the first therapeutic agent.

56. The formulation of claim 54, wherein the unit dose provides delayed release of at least a portion of the second therapeutic agent.

57. The formulation of claim 56, wherein the unit dose provides delayed release of substantially all of the second therapeutic agent.

58. The formulation of claim 53, wherein the unit dose is adapted to be administered to the patient within about 4 hours before bed.

59. The formulation of claim 58, wherein the unit dose is adapted to be administered to the patient within about 2 hours before bed, within about 1 hour of bed or substantially immediately before bed.

60. The formulation of claim 53, wherein the unit dose provides immediate release of at least a portion of the second therapeutic agent.

61. The formulation of claim 60, wherein the unit dose provides immediate release of substantially all of the second therapeutic agent.

62. The formulation of claim 60, wherein the unit dose provides delayed release of at least a portion of the first therapeutic agent.

63. The formulation of claim 62, wherein the unit dose provides delayed release of substantially all of the first therapeutic agent.

64. The formulation of claim 60, wherein the unit dose is adapted to be administered to the patient within about 4 hours of waking, within about 2 hours of waking, within about 1 hour of waking, before, with or after a meal.

65. A method of treating a disorder treatable by administration of a first therapeutic agent having 5HT2/5HT3 antagonist and alpha-2 antagonist activity, a second therapeutic agent, or both, wherein the second therapeutic agent possesses both serotonin reuptake inhibitory activity and norepinephrine reuptake inhibitory activity, the method comprising administering the first therapeutic agent to the patient, and within about 18 hours of administering the first therapeutic agent, administering the second therapeutic agent, wherein combined administration of the first therapeutic agent and the second therapeutic agent is effective to treat at least one disorder, wherein a reduction in at least one side effect associated with the first therapeutic agent, the second therapeutic agent, or both is obtained, and wherein at least one such side effect is selected from the group consisting of daytime sedation, nausea, emesis, weight gain and cognitive impairment.

66. The method of claim 65, wherein the first therapeutic agent comprises one or members of the group consisting of mirtazapine, mirtazapine enriched in either the R- or S-enantiomer, setiptiline or a combination thereof.

67. The method of claim 65, wherein the first therapeutic agent comprises mirtazapine.

68. The method of claim 65, wherein the first therapeutic agent comprises setiptiline.

69. The method of claim 65, wherein when the second therapeutic agent possesses serotonin reuptake inhibitory activity and norepinephrine reuptake inhibitory activity; and the ratio of serotonin reuptake inhibitory activity to norepinephrine reuptake inhibitory activity is in the range of about 1:50 to 50:1, about 1:20 to 20:1 or about 1:10 to 10:1.

70. The method of claim 65, wherein the second therapeutic agent comprises a serotonin norepinephrine reuptake inhibitor (SNRI).

71. The method of claim 70, wherein the SNRI is selected from the group consisting of duloxetine, venlafaxine, desvenlafaxine, indeloxazine and pharmaceutically acceptable salts, derivatives and combinations thereof.

72. The method of claim 65, wherein the second therapeutic agent comprises a norepinephrine serotonin reuptake inhibitor (NSRI).

73. The method of claim 72, wherein the NSRI is selected from the group consisting of milnacipran, derivatives of milnacipran, bicifadine and pharmaceutically acceptable salts, derivatives and combinations thereof.

74. The method of claim 73, wherein the NSRI is a racemic mixture of 1S,2R-milnacipran and 1R,2S-milnacipran or is milnacipran enriched in the 1S,2R-enantiomer of milnacipran, wherein a mass/mass ratio of the (1S,2R) enantiomer to the (1R,2S) enantiomer of milnacipran is greater than 1:1, greater than or equal to about 55:45, greater than or equal to about 60:40, greater than or equal to about 65:35, greater than or equal to about 70:30, greater than or equal to about 75:25, greater than or equal to about 80:20, greater than or equal to about 82:18, greater than or equal to about 84:16, greater than or equal to about 86:14, greater than or equal to about 88:12, greater than or equal to about 90:10, greater than or equal to about 91:9, greater than or equal to about 92:8, greater than or equal to about 93:7, greater than or equal to about 94:6, greater than or equal to about 95:5, greater than or equal to about 96:4, greater than or equal to about 97:3, greater than or equal to about 98:2, greater than or equal to about 99:1, greater than or equal to about 99.5:0.5, in a range of about 55:45 to about 95:5, in a range of about 55:45 to about 92.5:7.5, in a range of about 55:45 to about 90:10, in a range of about 60:40 to about 95:5, in a range of about 60:40 to about 92.5:7.5, in a range of about 60:40 to about 90:10, in a range of about 65:35 to about 95:5, in a range of about 65:35 to about 92.5:7.5, in a range of about 65:35 to about 90:10, in a range of about 60:30 to about 95:5, in a range of about 70:30 to about 92.5:7.5, in a range of about 70:30 to about 90:10.

75. The formulation of claim 65, wherein the second therapeutic agent comprises a reversible inhibitor of monoamine oxidase A (RIMA).

76. The formulation of claim 75, wherein the RIMA is selected from the group consisting of brofaromine, moclobemide, and pharmaceutically acceptable salts, derivatives and combinations thereof.

77. The method of claim 65, wherein the first and second therapeutic agents are administered in a unit dose.

78. The method of claim 77, wherein the unit dose provides immediate release of at least a portion of the first therapeutic agent.

79. The method of claim 78, wherein the unit dose provides immediate release of substantially all of the first therapeutic agent.

80. The method of claim 77, wherein the unit dose provides delayed release of at least a portion of the second therapeutic agent.

81. The method of claim 80, wherein the unit dose provides delayed release of substantially all of the second therapeutic agent.

82. The method of claim 77, wherein the unit dose is administered to the patient within about 4 hours before bed, within about 2 hours of bed, within about 1 hour of bed or substantially immediately before bed.

83. The method of claim 77, wherein the unit dose provides immediate release of at least a portion of the second therapeutic agent.

84. The method of claim 83, wherein the unit dose provides immediate release of substantially all of the second therapeutic agent.

85. The method of claim 83, wherein the unit dose provides delayed release of at least a portion of the first therapeutic agent.

86. The method of claim 85, wherein the unit dose provides delayed release of substantially all of the first therapeutic agent.

87. The method of claim 77, wherein the unit dose is administered to the patient within about 4 hours after waking, within about 2 hours after waking, within about 1 hour after waking, before, with or after a meal.

88. The method of claim 87, wherein the first therapeutic agent is administered before bed and the second therapeutic agent is administered after waking.

89. The method of claim 88, wherein the first therapeutic agent is administered within about 4 hours before bed, within about 2 hours before bed, within about 1 hour before bed or substantially immediately before bed.

90. The method of claim 87, wherein the second therapeutic agent is administered within about 4 hours of waking, within about 2 hours of waking, within about 1 hour of waking, before, with or after a meal.

91. The method of claim 65, wherein the method provides a reduction in two or more side effects selected from the group consisting of daytime sedation, cognitive impairment, iatrogenic weight gain, nausea and emesis.

92. The method of claim 65, wherein the disorder is selected from the group consisting of depression, schizophrenia, anxiety disorders, affective disorders, sleep-related breathing disorders, insomnia, migraine headache, chronic tension-type headache, hot flashes, chronic lower back pain, neuropathic pain (e.g. from diabetic peripheral neuropathy) and functional somatic syndromes.

93. The method of claim 92, wherein the disorder is an anxiety disorder selected from the group consisting of generalized anxiety disorder, panic disorder, phobias, and post-traumatic stress disorder.

94. The method of claim 92, wherein the disorder is a sleep-related breathing disorder selected from the group consisting of sleep apnea, sleep hypopnea, upper airway resistance syndrome, and snoring.

95. The method of claim 92, wherein the disorder is a functional somatic syndrome selected from the group consisting of fibromyalgia syndrome, chronic fatigue syndrome, and irritable bowel syndrome.

96. A kit comprising a first therapeutic agent, a second therapeutic agent and instructions for administering the first therapeutic agent before bed and the second therapeutic agent after waking, wherein the first therapeutic agent comprises a 5HT₂/5HT₃ antagonist/alpha-2 antagonist and the second therapeutic agent possesses both serotonin reuptake inhibitory activity and norepinephrine reuptake inhibitory activity or is a reversible inhibitor of monoamine oxidase A (RIMA).

97. The kit of claim 96, wherein the first therapeutic agent comprises one or members of the group consisting of mirtazapine, mirtazapine enriched in either the R- or S-enantiomer, setiptiline, or a combination thereof.

98. The kit of claim 97, wherein the 5HT₂/5HT₃ antagonist/alpha-2 antagonist comprises mirtazapine.

99. The kit of claim 97, wherein the 5HT₂/5HT₃ antagonist/alpha-2 antagonist comprises setiptiline.

100. The kit of claim 96, wherein when the second therapeutic agent possesses serotonin reuptake inhibitory activity and norepinephrine reuptake inhibitory activity; and the ratio of serotonin reuptake inhibitory activity to norepinephrine reuptake inhibitory activity is in the range of about 1:50 to 50:1, about 1:20 to 20:1 or about 1:10 to 10:1.

101. The kit of claim 96, wherein the second therapeutic agent comprises a serotonin norepinephrine reuptake inhibitor (SNRI).

102. The kit of claim 101, wherein the SNRI is selected from the group consisting of duloxetine, venlafaxine, desvenlafaxine, indeloxazine and pharmaceutically acceptable salts, derivatives and combinations thereof.

103. The kit of claim 96, wherein the second therapeutic agent comprises a norepinephrine serotonin reuptake inhibitor (NSRI).

104. The kit of claim 103, wherein the NSRI is selected from the group consisting of milnacipran, derivatives of milnacipran, bicifadine, and pharmaceutically acceptable salts, derivatives and combinations thereof.

105. The kit of claim 104, wherein the NSRI is a racemic mixture of 1S,2R-milnacipran and 1R,2S-milnacipran or is milnacipran enriched in the 1S,2R-enantiomer of milnacipran, wherein a mass/mass ratio of the (1S,2R) enantiomer to the (1R,2S) enantiomer of milnacipran is greater than 1:1, greater than or equal to about 55:45, greater than or equal to about 60:40, greater than or equal to about 65:35, greater than or equal to about 70:30, greater than or equal to about 75:25, greater than or equal to about 80:20, greater than or equal to about 82:18, greater than or equal to about 84:16, greater than or equal to about 86:14, greater than or equal to about 88:12, greater than or equal to about 90:10, greater than or equal to about 91:9, greater than or equal to about 92:8, greater than or equal to about 93:7, greater than or equal to about 94:6, greater than or equal to about 95:5, greater than or equal to about 96:4, greater than or equal to about 97:3, greater than or equal to about 98:2, greater than or equal to about 99:1, greater than or equal to about 99.5:0.5, in a range of about 55:45 to about 95:5, in a range of about 55:45 to about 92.5:7.5, in a range of about 55:45 to about 90:10, in a range of about 60:40 to about 95:5, in a range of about 60:40 to about 92.5:7.5, in a range of about 60:40 to about 90:10, in a range of about 65:35 to about 95:5, in a range of about 65:35 to about 92.5:7.5, in a range of about 65:35 to about 90:10, in a range of about 60:30 to about 95:5, in a range of about 70:30 to about 92.5:7.5, in a range of about 70:30 to about 90:10.

106. The kit of claim 96, wherein the second therapeutic agent comprises a reversible inhibitor of monoamine oxidase A (RIMA).

107. The kit of claim 106, wherein the second therapeutic agent comprises a RIMA selected from the group consisting of brofaromine, moclobemide, and pharmaceutically acceptable salts, derivatives and combinations thereof.

108. The kit of claim 97, wherein the kit comprises instructions to administer the first therapeutic agent within about 4 hours before bed, within about 2 hours before bed, within about 1 hour before or substantially immediately before bed.

109. The kit of claim 97, wherein the kit comprises instructions to administer the second therapeutic agent within about 4 hours of waking, within about 2 hours after waking, within about 1 hour after waking, before, with or after a meal.

110. A unit dosage form containing a synergistic combination of a first therapeutic agent and a second therapeutic agent, wherein the first therapeutic agent comprises a 5HT₂/5HT₃ antagonist/alpha-2 antagonist and the second therapeutic agent possesses both serotonin reuptake inhibitory activity and norepinephrine reuptake inhibitory activity norepinephrine or is a reversible inhibitor of monoamine oxidase A (RIMA).

111. The unit dosage of claim 110, wherein the unit dosage provides effective treatment of at least one disorder selected from the group consisting of depression, schizophrenia, anxiety disorders, affective disorders, sleep-related breathing disorders, insomnia, migraine headache, chronic tension-type headache, hot flashes, chronic lower back pain, neuropathic pain (e.g. from diabetic peripheral neuropathy) and functional somatic syndromes.

112. The unit dose of claim 111, wherein the disorder is an anxiety disorder selected from the group consisting of generalized anxiety disorder, panic disorder, phobias, and post-traumatic stress disorder.

113. The unit does of claim 111, wherein the disorder is a sleep-related breathing disorder selected from the group consisting of sleep apnea, sleep hypopnea, upper airway resistance syndrome, and snoring.

114. The unit dose of claim 111, wherein the disorder is a functional somatic syndrome selected from the group consisting of fibromyalgia syndrome, chronic fatigue syndrome, and irritable bowel syndrome.

115. The unit dose of claim 110, wherein the first therapeutic agent comprises one or members of the group consisting of mirtazapine, mirtazapine enriched in either the R- or S-enantiomer, setiptiline, a pharmaceutically acceptable salt of mirtazapine (or a stereoisomer thereof) and a pharmaceutically setiptiline (or a stereoisomer thereof).

116. The unit dose of claim 115, wherein the first therapeutic agent comprises mirtazapine.

117. The unit dose of claim 115, wherein the first therapeutic agent comprises setiptiline.

118. The unit dose of claim 110, wherein when the second therapeutic agent possesses serotonin reuptake inhibitory activity and norepinephrine reuptake inhibitory activity; and the ratio of serotonin reuptake inhibitory activity to norepinephrine reuptake inhibitory activity is in the range of about 1:50 to 50:1, about 1:20 to 20:1 or about 1:10 to 10:1.

119. The unit dose of claim 110, wherein the second therapeutic agent comprises a SNRI.

120. The unit dose of claim 119, wherein the SNRI is a member of the group consisting of duloxetine, venlafaxine, desvenlafaxine, indeloxazine and pharmaceutically acceptable salts, derivatives and combinations thereof.

121. The unit dose of claim 110, wherein the second therapeutic agent comprises a NSRI.

122. The unit dose of claim 121, wherein the NSRI is selected from the group consisting of milnacipran, derivatives of milnacipran, bicifadine, and pharmaceutically acceptable salts, derivatives and combinations thereof.

123. The method of claim 122, wherein the NSRI is a racemic mixture of 1S,2R-milnacipran and 1R,2S-milnacipran or is milnacipran enriched in the 1S,2R-enantiomer of milnacipran, wherein a mass/mass ratio of the (1S,2R) enantiomer to the (1R,2S) enantiomer of milnacipran is greater than 1:1, greater than or equal to about 55:45, greater than or equal to about 60:40, greater than or equal to about 65:35, greater than or equal to about 70:30, greater than or equal to about 75:25, greater than or equal to about 80:20, greater than or equal to about 82:18, greater than or equal to about 84:16, greater than or equal to about 86:14, greater than or equal to about 88:12, greater than or equal to about 90:10, greater than or equal to about 91:9, greater than or equal to about 92:8, greater than or equal to about 93:7, greater than or equal to about 94:6, greater than or equal to about 95:5, greater than or equal to about 96:4, greater than or equal to about 97:3, greater than or equal to about 98:2, greater than or equal to about 99:1, greater than or equal to about 99.5:0.5, in a range of about 55:45 to about 95:5, in a range of about 55:45 to about 92.5:7.5, in a range of about 55:45 to about 90:10, in a range of about 60:40 to about 95:5, in a range of about 60:40 to about 92.5:7.5, in a range of about 60:40 to about 90:10, in a range of about 65:35 to about 95:5, in a range of about 65:35 to about 92.5:7.5, in a range of about 65:35 to about 90:10, in a range of about 60:30 to about 95:5, in a range of about 70:30 to about 92.5:7.5, in a range of about 70:30 to about 90:10.

124. The unit dose of one of 110, wherein the second therapeutic agent comprises a reversible inhibitor of monoamine oxidase A (RIMA).

125. The unit dose of claim 124, wherein the second therapeutic agent comprises a RIMA selected from the group consisting of brofaromine, moclobemide, and pharmaceutically acceptable salts, derivatives and combinations thereof.

126. The unit dose of claim 110, wherein the unit dose provides immediate release of at least a portion of the first therapeutic agent.

127. The unit dose of claim 126, wherein the unit dose provides immediate release of substantially all of the first therapeutic agent.

128. The unit dose of claim 126, wherein the unit dose provides delayed release of at least a portion of the second therapeutic agent.

129. The unit dose claim 128, wherein the unit dose provides delayed release of substantially all of the second therapeutic agent.

130. The unit dose claim 126, wherein the unit dose is adapted to be administered to the patient within about 4 hours before bed, within about 2 hours before bed, within about 1 hour before bed or substantially immediately before bed.

131. The unit dose of claim 110, wherein the unit dose provides immediate release of at least a portion of the second therapeutic agent.

132. The unit dose of claim 130, wherein the unit dose provides immediate release of substantially all of the second therapeutic agent.

133. The unit dose of claim 130, wherein the unit dose provides delayed release of at least a portion of the first therapeutic agent.

134. The unit dose of claim 133, wherein the unit dose provides delayed release of substantially all of the first therapeutic agent.

135. The unit dose of claim 131, wherein the unit dose is administered to the patient within about 4 hours after waking, within about 2 hours after waking, within about 1 hour after waking, before, with or after a meal.

136. The unit dose of claim 110, wherein the unit dose comprises about 0.5-45 mg of mirtazapine and about 10 to about 400 mg of milnacipran.

137. The unit dose of claim 137, wherein the unit dose comprises about 0.5 to about 5 mg of mirtazapine and about 20 to about 200 of milnacipran.

138. The unit dose of claim 136, wherein the NSRI is a racemic mixture of 1S,2R-milnacipran and 1R,2S-milnacipran or is milnacipran enriched in the 1S,2R-enantiomer of milnacipran, wherein a mass/mass ratio of the (1S,2R) enantiomer to the (1R,2S) enantiomer of milnacipran is greater than 1:1, greater than or equal to about 55:45, greater than or equal to about 60:40, greater than or equal to about 65:35, greater than or equal to about 70:30, greater than or equal to about 75:25, greater than or equal to about 80:20, greater than or equal to about 82:18, greater than or equal to about 84:16, greater than or equal to about 86:14, greater than or equal to about 88:12, greater than or equal to about 90:10, greater than or equal to about 91:9, greater than or equal to about 92:8, greater than or equal to about 93:7, greater than or equal to about 94:6, greater than or equal to about 95:5, greater than or equal to about 96:4, greater than or equal to about 97:3, greater than or equal to about 98:2, greater than or equal to about 99:1, greater than or equal to about 99.5:0.5, in a range of about 55:45 to about 95:5, in a range of about 55:45 to about 92.5:7.5, in a range of about 55:45 to about 90:10, in a range of about 60:40 to about 95:5, in a range of about 60:40 to about 92.5:7.5, in a range of about 60:40 to about 90:10, in a range of about 65:35 to about 95:5, in a range of about 65:35 to about 92.5:7.5, in a range of about 65:35 to about 90:10, in a range of about 60:30 to about 95:5, in a range of about 70:30 to about 92.5:7.5, in a range of about 70:30 to about 90:10.

139. The unit dose of claim 110, wherein the unit dose comprises about 0.5-45 mg of mirtazapine and about 5 to 100 mg of duloxetine or a pharmaceutically acceptable salt or stereoisomer thereof.

140. The unit dose of claim 139, wherein the unit dose comprises about 0.5 to about 5 mg of mirtazapine and about 10 to about 80 mg of duloxetine or a pharmaceutically acceptable salt or stereoisomers thereof.

141. The unit dose of claim 110, wherein the unit dose contains less than 100% of the average effective dose of 5HT₂/5HT₃ antagonist/alpha-2 antagonist and less than 100% of the average effective dose of SNRI, NSRI or RIMA.

142. The unit dose of claim 110, wherein the unit dose contains less than about 75% of the average effective dose of 5HT₂/5HT₃ antagonist/alpha-2 antagonist and less than 75% of the average effective dose of the second therapeutic agent.

143. The unit dose of claim 110, wherein the unit dose contains only about 0.5 to 161% of the average effective dose of 5HT₂/5HT₃ antagonist/alpha-2 antagonist and about 0.5 to 161% of the average effective dose the second therapeutic agent.
